(19) United States
(12) Patent Application Publication
(10) Pub. No.: US 2017/0143611 A1
(43) Pub. Date: May 25, 2017

(54) AGENTS AND METHOD FOR CLEANING AND/OR CARING FOR DAMAGED KERATIN FIBERS

(71) Applicant: Henkel AG & Co. KGaA, Duesseldorf (DE)

(72) Inventors: Thomas Hippe, Appen (DE); Astrid Kleen-Fehres, Hamburg (DE); Hartmut Manneck, Barnitz (DE)

(73) Assignee: Henkel AG & Co. KGaA, Duesseldorf (DE)

(21) Appl. No.: 15/352,683

(22) Filed: Nov. 16, 2016

(30) Foreign Application Priority Data
Nov. 24, 2015 (DE) 10 2015 223 196.8

(51) Int. Cl. A61K 8/81; A61Q 5/02; A61Q 5/12; A61K 8/362; A61K 8/44

(52) U.S. Cl. CPC A61K 8/8176; A61K 8/362; A61K 8/44; A61Q 5/12; A61Q 5/02; A61K 2800/30

(57) ABSTRACT

The invention relates to agents for cleaning and/or caring for damaged keratin fibers, including a combination of at least one dicarboxylic acid having 2 to 10 carbon atoms and at least one selected amino acid, and a method for cleaning and/or caring for keratin fibers by means of such agents.

### FIELD OF THE INVENTION

The present invention generally relates to agents for cleaning and/or caring for damaged keratin fibers, including a combination of at least one dicarboxylic acid having 2 to 10 carbon atoms and at least one selected amino acid, and a method for cleaning and/or caring for keratin fibers by means of such agents.

### BACKGROUND OF THE INVENTION

In everyday life, great importance is attached to keratin fibers, particularly hair, as a fixed component of the human body and as an important component of human clothing and home textiles. Over time, treatment with washing, cleaning, styling, and dyeing products for cleaning and styling purposes and exposure of the keratin fibers to environmental influences such as ozone, salt water, chlorinated water, and IR, UV, and thermal radiation (blow drying) lead to cumulative damage to the fibers and therefore to a reduction in the quality of the fibers. For example, both the cleaning of hair with shampoos and the decorative styling of the hair by dyeing or perming are actions that influence the natural structure and the properties of the hair. Therefore, the wet and dry combability, hold, body, shine, and feel of the hair, for example, can be unsatisfactory after such a treatment. Furthermore, in the case of dyed hair, the hold of the dye on the hair can be unsatisfactory particularly if the hair is washed frequently, causing the dye to gradually bleed out.

Not least because of the heavy stress on the hair, for example due to dyeing, perming, cleaning of the hair with shampoos, and environmental stresses, the importance of care products having a sufficiently strong effect that lasts as long as possible is increasing. Such care agents influence the natural structure and the properties of the hair. In this way, the wet and dry combability, hold, and body of the hair can be improved or the hair can be protected from an increased rate of split ends, for example, after such treatments.

Therefore, it has long been common to subject the hair to a specific aftertreatment. In such an aftertreatment, the hair is treated with special active substances, such as quaternary ammonium salts or special polymers, usually in the form of a conditioning agent. The combability, hold, and body of the hair are improved and the rate of split ends is reduced by means of this treatment, depending on the formulation.

Care additives and film formers are often also added to perming agents, but do not considerably improve the hair structure. For example, high-molecular-weight polymers that attach to the topmost layer of the skin and the hair and that produce an external, subjectively perceptibly improved texture of the hair there are used for this purpose. However, the structural damage inside the hair, which is caused especially by the reduction process during perming, cannot thereby be reduced, because the substances cannot penetrate into the hair because of the size of the substances. In addition, the lastingness of the effects of the structure-improving additives is often unsatisfactory, because said additives only superficially adhere to the hair.

There have been attempts to remedy this problem by polymerizing monomeric compounds directly on the hair. According to the teaching of U.S. Pat. No. 5,362,486, certain urethane oligomers having terminal bisulfite or acrylate groups are applied to the hair and then polymerized, wherein adhering polymers are formed in situ on the hair. By means of this method, the surface properties of hair are favorably influenced, such as volume, shine, hold, combability, and resistance to the take-up of moisture and air contaminants and to the loss of hair dyes. In the method, a radical polymerization occurs on the hair, i.e., the hair can with radical formers such as benzoyl peroxide.

Patent application WO 2005/115314 A1 discloses a method for restructuring keratin fibers, wherein the keratin fibers are brought into contact with cystine and with at least one dicarboxylic acid having 2 to 10 carbon atoms, wherein preferred dicarboxylic acids are selected from oxalic acid, malonic acid, succinic acid, glutaric acid, adipic acid, pimelic acid, azelaic acid, maleic acid, fumaric acid, and sorbic acid and succinic acid is especially preferred. Patent application DE 10051774 A1 describes the use of short-chain carboxylic acids having a molecular weight under 750 g/mol in cosmetic agents as an active substance for restructuring keratin fibers. Patent application EP 1174112 A discloses hair treatment agents that include an organic solvent, a cationic surfactant, and a higher alcohol as further mandatory constituents in addition to an organic acid and that are used to repair pores in hair.

The problem addressed by the invention is that of providing a method for restructuring keratin fibers that has advantages over the prior art and enables sufficient effectiveness and duration of effect. The method should be performable under conditions that are gentle on the fibers and should also be physiologically harmless, e.g., should get by without the use of reactive monomers.

Surprisingly, it has been found that the tensile elongation properties of keratin fibers can be improved and the strength thereof increased by means of a combination of at least one saturated dicarboxylic acid having 2 to 10 carbon atoms and at least one selected amino acid of formula (VI), as explained below. In the context of the present invention, it has been found that, by using agents, compositions, and methods according to the invention, the internal and external structure of keratin fibers can be changed, i.e., a restructuring of keratin fibers is enabled. In the sense of the present invention, the term “restructuring” is understood to mean, in particular, fiber strengthening, an increase in tearing force, and/or a reduction in the damage to keratin fibers caused by a wide range of influences. The restoration of the natural strength, for example, plays an important role here. Restructured fibers can be distinguished by, for example, an increased melting point (measured by means of DSC), increased tearing force, increased strength, increased elasticity, and/or increased volume, which, for example in the case of a hairstyle, can be exhibited in greater body. Furthermore, restructured fibers can have improved shine and/or improved texture and/or be easier to comb.

According to the invention, the term “keratin fibers” should be understood to mean pelts, wool, feathers, silk, and hair, particularly human hair.

According to the invention, the term “cleaning agents” should be understood to mean shampoos, shower preparations, washing creams, and make-up removers.

According to the invention, the term “care agents” should be understood to mean hair conditioners, hair conditioning agents, hair masks, leave-on conditioners, and hair care sprays.

The problem addressed by the invention is solved by means of agents, compositions, and methods according to the claims.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic agent for cleaning and/or caring for keratin fibers, particularly human hair, which is free of peroxide compounds and includes at least one saturated dicarboxylic acid having 2 to 10 carbon atoms and/or at least one salt of said acid(s), at least one amino acid of formula (VI)

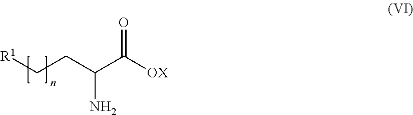

wherein X represents a hydrogen atom or a mono- or divalent cation; n represents 0, 1, 2, or 3; R¹ represents a residue that is selected from an amino group, a guanidine group, a (1H-imidazol-4-yl) group, a carboxylic acid amide group —CONH₂, a 1H-indol-3-yl group, a thiol group —SH, and a methylsulfanyl group —SCH₃, or at least one salt of said amino acid, and at least one cleaning and/or care active substance, selected from surfactants, fatty alcohols, and oils.

A method for cleaning and/or caring for keratin fibers, particularly human hair, that comprises the following steps: I. providing a composition (A), including at least one saturated dicarboxylic acid having 2 to 10 carbon atoms in a total amount of 2 to 20 wt %, preferably 5 to 15 wt %, especially preferably 8 to 12 wt %, converted to the undissociated acid and with respect to the weight of the composition (A), wherein the dicarboxylic acid is preferably selected from succinic acid, malic acid, oxalic acid, malonic acid, adipic acid, pimelic acid, suberic acid, azelaic acid, sebacic acid, and mixtures of these acids, wherein succinic acid and malic acid are especially preferred and succinic is extraordinarily preferred; at least one amino acid of formula (VI)

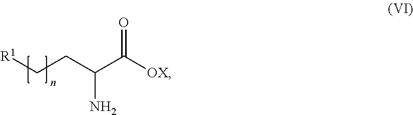

wherein X represents a hydrogen atom or a mono- or divalent cation; n represents 0, 1, 2, or 3; R¹ represents a residue that is selected from an amino group, a guanidine group, a (1H-imidazol-4-yl) group, a carboxylic acid amide group —CONH₂, a 1H-indol-3-yl group, a thiol group —SH, and a methylsulfanyl group —SCH₃, or at least one salt of said amino acid; and/or one salt thereof in a total amount of 0.4 to 7.0 wt %, preferably 0.8 to 5.0 wt %, especially preferably 1.5 to 4.0 wt %, converted to the undissociated acid and with respect to the weight of the composition (A), wherein preferably at least one of the amino acids arginine, histidine, or lysine and/or one salt thereof is included in a total amount of 0.4 to 7.0 wt %, preferably 0.8 to 5.0 wt %, especially preferably 1.5 to 4.0 wt %, converted to the undissociated acid and with respect to the weight of the composition (A); and water, preferably in an amount of 50 to 92 wt %, especially preferably 60 to 87 wt %, and extraordinarily preferably 65 to 80 wt %, with respect to the weight of the composition (A); optionally also at least one polymer A, which has at least ten constitutive units of formula (I);

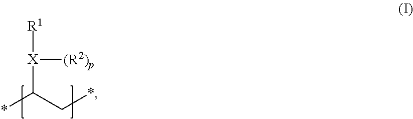

wherein X represents nitrogen or oxygen; R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group; and p=0 if X represents oxygen and p=1 if X represents nitrogen, wherein the polymer A does not include any permanently ionic constitutive units, wherein the at least one polymer A is preferably included in a total amount of 0.5 to 14 wt %, preferably 1.0 to 11 wt %, especially preferably 2.0 to 10 wt %, with respect to the weight of the composition (A), wherein the composition (A) preferably has a pH value in the range of 3.5 to 7.1, preferably 4.5 to 6.5, especially preferably 5.0 to 6.0, measured at 20° C.; water; and optionally also at least one substance selected from compounds of general formula (III),

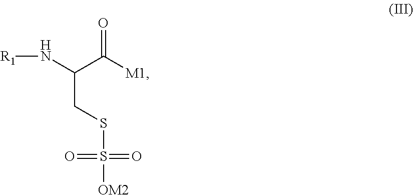

wherein R1 represents a hydrogen atom or a structural element of formula (IV),

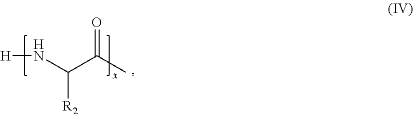

wherein x represents an integer from 1 to 100, the residue R2 in each of the structural elements of formula (IV) can be selected independently of the preceding structural element of formula (IV), R2 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group, M1 represents the group —OM2 or a structural element of formula (V),

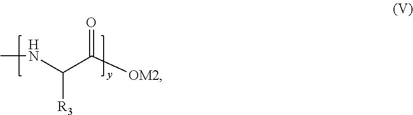

wherein y represents an integer from 1 to 100; the residue R3 in each of the structural elements of formula (V) can be selected independently of the preceding structural element of formula (V); R3 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group; M2 represents a hydrogen atom, an equivalent of a mono- or polyvalent cation, or an ammonium ion (NH₄)⁺; wherein one or more compounds of formula (III) stated above are preferably included in a total amount of 0.01 to 2.5 wt %, more preferably 0.05 to 0.5 wt %, and especially preferably 0.1 to 0.3 wt %, with respect to the weight of the composition (A); and polymers A, which have at least ten constitutive units of formula (I),

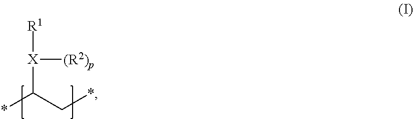

wherein X represents nitrogen or oxygen; R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group; and p=0 if X represents oxygen and p=1 if X represents nitrogen, wherein the polymer A does not include any permanently ionic constitutive units, wherein the at least one polymer A is preferably included in a total amount of 0.5 to 14 wt %, preferably 1.0 to 11 wt %, especially preferably 2.0 to 10 wt %, with respect to the weight of the composition (A); II. providing a cleaning and/or care composition (B), including at least one cleaning and/or care active substance, selected from surfactants, fatty alcohols, and oils; optionally water; and III. mixing the compositions (A) and (B) with each other, and, directly thereafter; IV. applying the mixture of (A) and (B) to the keratin fibers, particularly the human hair; V. optionally rinsing out after an exposure time of 0.1 to 60 minutes, preferably 0.5 to 15 minutes, especially preferably 1 to 10 minutes; and VI. optionally performing further hair treatments, such as conditioning and/or drying.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention relates first to a cosmetic agent for cleaning and/or caring for keratin fibers, particularly human hair, that is free of peroxide compounds, including
a) at least one saturated dicarboxylic acid having 2 to 10 carbon atoms and/or at least one salt of said acid(s),
b) at least one amino acid of formula (VI),

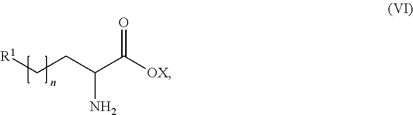

wherein
X represents a hydrogen atom or a mono- or divalent cation;
n represents 0, 1, 2, or 3;
R¹ represents a residue that is selected from an amino group, a guanidine group, a (1H-imidazol-4-yl) group, a carboxylic acid amide group —CONH₂, a 1H-indol-3-yl group, a thiol group —SH, and a methylsulfanyl group —SCH₃, or at least one salt of said amino acid, and
c) at least one cleaning and/or care active substance, selected from surfactants, fatty alcohols, and oils.

Saturated Dicarboxylic Acids having 2 to 10 Carbon Atoms and/or at Least One Salt of Said Acid(s)

Saturated dicarboxylic acids having 2 to 10 carbon atoms that are preferred according to the invention are selected from succinic acid, malic acid, oxalic acid, malonic acid, adipic acid, pimelic acid, suberic acid, azelaic acid, sebacic acid, and mixtures of said acids. Succinic acid and malic acid and mixtures thereof are especially preferred according to the invention. Succinic acid is extraordinarily preferred according to the invention. The mentioned dicarboxylic acids make a significant contribution to the restructuring effect of the cleaning and/or care agents according to the invention.

The optically active malic acid can be used in the form of D-malic acid or L-malic acid or as racemic DL-malic acid. DL-malic acid is preferred for cost reasons.

Depending on the pH value of the cleaning and/or care agent according to the invention or of the composition (A) used in one of the cleaning and/or care methods according to the invention, the at least one saturated dicarboxylic acid having 2 to 10 carbon atoms can exist as an undissociated acid or in a state of partial or complete dissociation. If the at least one saturated dicarboxylic acid having 2 to 10 carbon atoms exists in a state of partial or complete dissociation, the counterion is selected from physiologically acceptable cations, in particular the alkali metal, alkaline-earth metal, and zinc ions, ammonium ions, alkylammonium ions, alkanolammonium ions, and glucammonium ions, particularly the mono-, di-, and trimethyl-, -ethyl-, and -hydroxyethylammonium ions. Likewise preferred are the salts of the saturated dicarboxylic acids having 2 to 10 carbon atoms with amino-C₁-C₆-alkanols, particularly with monoethanolamine, and with amino-C₁-C₆-alkanediols, particularly with 2-amino-2-methylpropan-1-ol, 2-amino-2-methylpropan-1,3-diol, 2-aminopropan-1-ol, 3-aminopropan-1-ol, 1-aminopropan-2-ol (MIPA), and 2-amino-2-(hydroxymethyl)propane-1,3-diol (TRIS), wherein the salts with monoethanolamine, 2-amino-2-methylpropan-1-ol, and 2-amino-2-methylpropan-1,3-diol are especially preferred.

Sodium, potassium, magnesium, ammonium, and monoethanolammonium ions are extraordinarily preferred as counterions for the partially or completely dissociated saturated dicarboxylic acids having 2 to 10 carbon atoms. Apart therefrom, however, saturated dicarboxylic acid having 2 to 10 carbon atoms that are neutralized by means of basically reacting amino acids, such as arginine, lysine, ornithine, and histidine, can also be used.

The sodium, potassium, ammonium, monoethanolammonium, lysine, and arginine salts and mixtures thereof are preferred salts of the saturated dicarboxylic acids having 2 to 10 carbon atoms.

Preferred cleaning and/or care agents according to the invention include the at least one saturated dicarboxylic acid having 2 to 10 carbon atoms or one or more salts thereof in a total amount of 0.05 to 6 wt %, preferably 0.1 to 4 wt %, especially preferably 0.5 to 2.5 wt %, converted to the undissociated acid and with respect to the weight of the cleaning and/or care agent.

Even if the dicarboxylic acids exist in salt form, the amount specifications above relate to the particular dicarboxylic acid in undissociated form so that the amount specification is not falsified as a result of different molecular weights of the salts. For example, a weighing in of 15 wt % of disodium succinate hexahydrate would result in a concentration of succinic acid 6.55 wt % (converted).

Amino Acid of Formula (VI)

The restructuring effect of the cleaning and/or care agents according to the invention can essentially be traced back to the aforementioned dicarboxylic acids in interaction with at least one selected amino acid of formula (VI).

Therefore, the cleaning and/or care agents according to the invention include, as a further obligatory component, at least one amino acid of formula (VI),

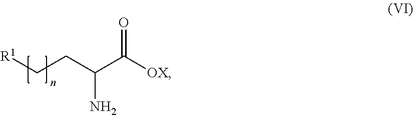

wherein
X represents a hydrogen atom or a mono- or divalent cation;
n represents 0, 1, 2, or 3;
R¹ represents a residue that is selected from an amino group, a guanidine group, a (1H-imidazol-4-yl) group, a carboxylic acid amide group —CONH₂, a 1H-indol-3-yl group, a thiol group —SH, and a methylsulfanyl group —SCH₃, or at least one salt of said amino acid.

Preferred amino acids of formula (VI) are selected from arginine, lysine, histidine, asparagine, glutamine, cysteine, methionine, tryptophan, and mixtures thereof. Cleaning and/or care agents especially preferred according to the invention include mixtures of arginine and lysine or at least one salt of these amino acids.

Preferred cleaning and/or care agents according to the invention include the at least one amino acid of formula (VI) or one or more salts thereof in a total amount of 0.05 to 3 wt %, preferably 0.1 to 2.5 wt %, especially preferably 0.3 to 1.2 wt %, converted to the undissociated acid and with respect to the weight of the cleaning and/or care agent. Other especially preferred cleaning and/or care agents according to the invention include mixtures of arginine and lysine or at least one salt of these amino acids in a total amount of 0.05 to 3 wt %, preferably 0.1 to 2.5 wt %, especially preferably 0.3 to 1.2 wt %, converted to the undissociated acid and with respect to the weight of the cleaning and/or care agent.

Cleaning and Care Active Substances

Surfactants

Cleaning active substances according to the invention are surfactants and emulsifiers. The terms “surfactants” and “emulsifiers” are understood to mean interface-active substances that can form adsorption layers at surfaces and interfaces or can aggregate to form association colloids or lyotropic mesophases in volume phases. Basic properties of surfactants and emulsifiers are the oriented absorption at interfaces, the aggregation into micelles, and the formation of lyotropic phases. A distinction is made between anionic surfactants consisting of a hydrophobic residue and a negatively charged hydrophilic headgroup, amphoteric surfactants, which bear both a negative charge and a compensating positive charge, cationic surfactants, which have a positively charged hydrophilic group in addition to a hydrophobic residue, and nonionic surfactants, which have no charges but instead have strong dipole moments and are strongly hydrated in aqueous solution.

Cleaning and/or care agents preferred according to the invention include at least one surfactant in a total amount of 0.1 to 30 wt %, preferably 0.5 to 20 wt %, especially preferably 1 to 15 wt %, extraordinarily preferably 3 to 10 wt %, further extraordinarily preferably 5 to 7 wt %, with respect to the weight of the cleaning and/or care agent.

In principle, all anionic surface-active substances suitable for use on the human body are suitable as anionic surfactants in cleaning and/or care agents according to the invention. These are characterized by a water-solubilizing anionic group, such as a carboxylate, sulfate, sulfonate, or phosphate group, and a lipophilic alkyl group having approximately 8 to 30 C atoms. In addition, glycol or polyglycol ether groups, ester, ether, and amide groups, and hydroxyl groups can be included in the molecule. Examples of suitable anionic surfactants are, in each case in the form of the sodium, potassium, ammonium, and mono-, di-, and trialkanolammonium salts having 2 to 4 C atoms in the alkanol group,
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,
mixtures of surface-active hydroxysulfonates according to application DE 3725030,
sulfated hydroxyalkyl polyethylene glycol ethers and/or hydroxyalkylene propylene glycol ethers according to application DE 3723354,
sulfonates of unsaturated fatty acids having 8 to 24 C atoms and 1 to 6 double bonds according to application DE 3926344,
esters of tartaric acid and citric acid with alcohols that are products of the addition of approximately 2-15 molecules of ethylene oxide and/or propylene oxide to fatty alcohols having 8 to 22 C atoms,
alkyl and/or alkenyl ether phosphates of formula (II),

R¹(OCH₂CH₂)ₙ—O—P(O)(OX)(OR²) (II),
in which R¹ preferably represents an aliphatic hydrocarbon residue having 8 to 30 carbon atoms, R² represents hydrogen, a residue (CH₂CH₂O)ₙR¹, or X, n represents numbers from 1 to 10, and X represents hydrogen, an alkali metal or alkaline-earth metal, or NR³R⁴R⁵R⁶, with R³ to R⁶ representing, independently of each other, hydrogen or a C1 to C4 hydrocarbon residue,
sulfated fatty acid alkylene glycol esters of formula (XII),

R⁷CO(AlkO)ₙSO₃M (XII)
in which R⁷CO— represents a linear or branched, aliphatic, saturated and/or unsaturated acyl residue having 6 to 22 C atoms, Alk represents CH₂CH₂, CHCH₃CH₂, and/or CH₂CHCH₃, n represents numbers from 0.5 to 5, and M represents a cation, which are described in DE 19736906,
monoglyceride sulfates and monoglyceride ether sulfates of formula (XIII),

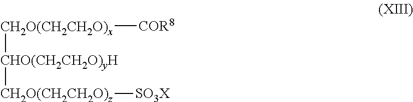

in which R⁸CO represents a linear or branched acyl residue having 6 to 22 carbon atoms, x, y, and z represent, in total, 0 or numbers from 1 to 30, preferably 2 to 10, and X represents an alkali metal or alkaline-earth metal. Typical examples of monoglyceride (ether) sulfates suitable according to the invention are the products of the reaction of lauric acid monoglyceride, coconut fatty acid monoglyceride, palmitic acid monoglyceride, stearic acid monoglyceride, oleic acid monoglyceride, and tallow fatty acid monoglyceride and ethylene oxide adducts thereof with sulfur trioxide or chlorosulfuric acid in the form of the sodium salts thereof. Monoglyceride sulfates of formula (XIII), in which R⁸CO represents a linear acyl residue having 8 to 18 carbon atoms, which have been described, for example, in EP 0561825 B1, EP 10561999 B1, DE 4204700 A1, and by A. K. Biswas et al. in J. Am. Oil Chem. Soc. 37, 171 (1960) and F. U. Ahmed in J. Am. Oil Chem. Soc. 67, 8 (1990), are preferably used,
amide ether carboxylic acids, which are described in EP 0690044,
products of the condensation of C₈-C₃₀ fatty acids with amino acids, such as sodium cocoyl glutamate, and/or with protein hydrolysates, which are known to a person skilled in the art as protein fatty acid condensates, for example.

Preferred anionic surfactants are alkyl polyglycol ether sulfates and ether carboxylic acids having, in each case, 10 to 18 C atoms in the alkyl group and 2 to 6 ethylene oxide groups in the molecule, acyl isethionates having 8 to 24 C atoms in the acyl group, and acylated amino acids and protein fatty acid condensates having, in each case, 8 to 24 C atoms in the acyl group, and mixtures of these anionic surfactants.

Cleaning agents preferred according to the invention include at least one anionic surfactant in a total amount of 0.1 to 30 wt %, preferably 0.5 to 20 wt %, especially preferably 1 to 15 wt %, extraordinarily preferably 3 to 10 wt %, further extraordinarily preferably 5 to 7 wt %, with respect to the weight of the cleaning agent. Care agents preferred according to the invention also include at least one anionic surfactant, but in smaller total amounts of 0.01 to 10 wt %, preferably 0.1 to 5 wt %, especially preferably 0.5 to 3 wt %, with respect to the weight of the care agent.

Surface-active compounds that bear at least one quaternary ammonium group and at least one —COO⁽⁻⁾ or —SO₃⁽⁻⁾ group in the molecule are referred to as zwitterionic surfactants. Especially suitable zwitterionic surfactants are the betaines such as the N-alkyl-N,N-dimethylammonium glycinates, for example coco alkyl dimethyl ammonium glycinate, N-acyl-aminopropyl-N,N-dimethylammonium glycinates, for example cocoacyl aminopropyl dimethyl ammonium glycinate, and 2-alkyl-3-carboxymethyl-3-hydroxyethyl-imidazolines each having 8 to 18 C atoms in the alkyl or acyl group, and cocoacyl aminoethyl hydroxyethyl carboxymethyl glycinate. A preferred zwitterionic surfactant is the fatty acid amide derivative known under the INCI name Cocamidopropyl Betaine.

Cleaning agents preferred according to the invention include at least one zwitterionic surfactant in a total amount of 0.1 to 15 wt %, preferably 0.2 to 10 wt %, especially preferably 0.5 to 8 wt %, extraordinarily preferably 1 to 5 wt %, further extraordinarily preferably 2 to 4 wt %, with respect to the weight of the cleaning agent. Care agents preferred according to the invention also include at least one zwitterionic surfactant, but in smaller total amounts of 0.01 to 7 wt %, preferably 0.1 to 5 wt %, especially preferably 0.1 to 3 wt %, with respect to the weight of the care agent.

The term “ampholytic surfactants” is understood to mean surface-active compounds that include at least one free amino group and at least one —COOH or —SO₃H group in addition to a C₈-C₂₄ alkyl or acyl group in the molecule and are capable of forming inner salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids each having 8 to 24 C atoms in the alkyl group. Especially preferred ampholytic surfactants are N-cocoalkylaminopropionate, cocoacylaminoethylaminopropionate, and C₁₂-C₁₈ acyl sarcosine.

Cleaning agents preferred according to the invention include at least one ampholytic surfactant in a total amount of 0.1 to 10 wt %, preferably 0.2 to 8 wt %, especially preferably 0.5 to 6 wt %, extraordinarily preferably 1 to 5 wt %, further extraordinarily preferably 2 to 4 wt %, with respect to the weight of the cleaning agent. Care agents preferred according to the invention also include at least one ampholytic surfactant, but in total amounts of 0.01 to 7 wt %, preferably 0.1 to 5 wt %, especially preferably 0.1 to 3 wt %, with respect to the weight of the care agent.

Nonionic surfactants include preferably a polyol group, one or more polyalkylene glycol ether groups, particularly one or more polyethylene glycol ether groups, or a combination of polyol group and polyglycol ether group as a hydrophilic group. Preferred nonionic surfactants are, for example,
products of the addition of 2 to 50 mol of ethylene oxide and/or 0 to 5 mol of propylene oxide to linear and branched fatty alcohols having 8 to 30 C atoms, to fatty acids having 8 to 30 C atoms, and to alkylphenols having 8 to 15 C atoms in the alkyl group,
products of the addition of 2 to 50 mol of ethylene oxide and/or 0 to 5 mol of propylene oxide to linear and branched fatty alcohols having 8 to 30 C atoms, to fatty acids having 8 to 30 C atoms, and to alkylphenols having 8 to 15 C atoms in the alkyl group, said products being end-capped with a methyl residue or C2-C6 alkyl residue,
C₁₂-C₃₀ fatty acid mono- and diesters of products of the addition of 1 to 30 mol of ethylene oxide to glycerol,
products of the addition of 5 to 60 mol of ethylene oxide to castor oil and hardened castor oil,
alkoxylated, particularly ethoxylated, triglycerides,
alkoxylated fatty acid alkyl esters of formula (XIV),

R¹CO—(OCH₂CHR²)_{w}OR³ (XIV),
in which R¹CO represents a linear or branched, saturated and/or unsaturated acyl residue having 6 to 22 carbon atoms, R² represents hydrogen or methyl, R³ represents linear or branched alkyl residues having 1 to 4 carbon atoms, and w represents numbers from 1 to 20,
amine oxides,
hydroxy mixed ethers, which are described, for example, in laid-open application DE 19738866,
sorbitan fatty acid esters and products of the addition of ethylene oxide to sorbitan fatty acid esters, such as the polysorbates,
sugar fatty acid esters and products of the addition of ethylene oxide to sugar fatty acid esters,
products of the addition of ethylene oxide to fatty acid alkanolamides and fatty amines,
sugar surfactants of the type of the alkyl and alkenyl oligoglycosides according to formula (XV),

R⁴O-[G]ₚ (XV)
in which R⁴ represents an alkyl or alkenyl residue having 4 to 22 carbon atoms, G represents a sugar residue having 5 or 6 carbon atoms, and p represents numbers from 1 to 10. They can be obtained according to the relevant methods of preparative organic chemistry.

The alkyl and alkenyl oligoglycosides can be derived from aldoses or ketoses having 5 or 6 carbon atoms, preferably from glucose. The preferred alkyl and/or alkenyl oligoglycosides are therefore alkyl and/or alkenyl oligoglucosides. The index number p in general formula (XV) indicates the degree of oligomerization, i.e., the distribution of mono- and oligoglycosides, and represents a number between 1 and 10. While p must always be an integer in the individual molecule, and can assume especially the values p=1 to 6 here, the value p for a certain alkyl oligoglycoside is an analytically determined calculated value, which is usually a rational number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and in particular between 1.2 and 1.4 are preferred from the perspective of application. The alkyl or alkenyl residue R⁴ can be derived from primary alcohols having 4 to 11, preferably 8 to 10 carbon atoms. Typical examples are butanol, caproic alcohol, capryl alcohol, capric alcohol, and undecyl alcohol, and technical mixtures thereof, which are obtained, for example, in the hydrogenation of technical fatty acid methyl esters or in the course of the hydrogenation of aldehydes from Roelen oxo synthesis. Alkyl oligoglucosides of chain length C₈-C₁₀ (degree of oligomerization=1 to 3) that arise as a forerun in the separation by distillation of technical C₈-C₁₈ coconut fatty alcohol and can be contaminated with a fraction of less than 6 wt % of C₁₂ alcohol, and alkyl oligoglucosides based on technical C_{9/11} oxo alcohols (degree of oligomerization=1 to 3), are preferred. The alkyl or alkenyl residue R¹⁵ can also be derived from primary alcohols having 12 to 22, preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical mixtures thereof, which can be obtained as described above. Alkyl oligoglucosides based on hardened C_{12/14} coconut alcohol having a degree of oligomerization of 1 to 3 are preferred.
sugar surfactants of the type of the fatty acid N-alkyl polyhydroxyalkyl amides, a nonionic surfactant of formula (VII),

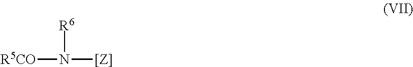

in which R⁵CO represents an aliphatic acyl residue having 6 to 22 carbon atoms, R⁶ represents hydrogen or an alkyl or hydroxyalkyl residue having 1 to 4 carbon atoms, and [Z] represents a linear or branched polyhydroxyalkyl residue having 3 to 12 carbon atoms and 3 to 10 hydroxyl groups. The fatty acid N-alkyl polyhydroxyalkyl amides are known substances that can be obtained typically by the reductive amination of a reducing sugar with ammonia, an alkylamine, or an alkanolamine and subsequent acylation with a fatty acid, a fatty acid alkyl ester, or a fatty acid chloride. In regard to the methods for the production thereof, reference is made to US patent documents U.S. Pat. No. 1,985,424, U.S. Pat. No. 2,016,962, and U.S. Pat. No. 2,703,798 and WO 92/06984. The fatty acid N-alkyl polyhydroxyalkyl amides are preferably derived from reducing sugars having 5 or 6 carbon atoms, particularly from glucose. The preferred fatty acid N-alkyl polyhydroxyalkyl amides are therefore the fatty acid N-alkyl glucamides represented by formula (VIII):

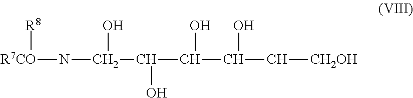

Glucamides of formula (VIII), in which R⁸ represents hydrogen or an alkyl group and R⁷CO represents the acyl residue of caproic acid, caprylic acid, capric acid, lauric acid, myristic acid, palmitic acid, palmitoleic acid, stearic acid, isostearic acid, oleic acid, elaidic acid, petroselinic acid, linoleic acid, linolenic acid, arachidic acid, gadoleic acid, behenic acid, or erucic acid or technical mixtures thereof, are preferably used as fatty acid N-alkyl polyhydroxyalkyl amides. Especially preferred are fatty acid N-alkyl glucamides of formula (VIII) that are obtained by the reductive amination of glucose with methylamine and subsequent acylation with lauric acid or C12/14 coconut fatty acid or with a corresponding derivative. Furthermore, the polyhydroxyalkyl amides can also be derived from maltose and Palatinose.

The products of the addition of alkylene oxide to saturated linear fatty alcohols and fatty acids each having 2 to 30 moles of ethylene oxide per mole of fatty alcohol or fatty acid have proven to be preferred nonionic surfactants. Preparations having excellent properties are likewise obtained if said preparations include fatty acid esters of ethoxylated glycerol as nonionic surfactants.

These compounds are characterized by the following parameters. The alkyl residue R includes 6 to 22 carbon atoms and can be both linear and branched. Primary linear aliphatic residues and aliphatic residues that are methyl-branched in the 2 position are preferred. Such alkyl residues are, for example, 1-octyl, 1-decyl, 1-lauryl, 1-myristyl, 1-cetyl, and 1-stearyl. 1-Octyl, 1-decyl, 1-lauryl, and 1-myristyl are especially preferred. If “oxo alcohols” are used as starting substances, compounds having an odd number of carbon atoms in the alkyl chain predominate.

Furthermore, the sugar surfactants are exceedingly preferred nonionic surfactants. These can be included in the cleaning and/or care agents according to the invention preferably in amounts of 0.1-20 wt %, with respect to the entire agent. Amounts of 0.5-15 wt % are preferred. Amounts of 0.5-7.5 wt % are exceedingly preferred.

The compounds having alkyl groups used as a surfactant can be uniform substances. However, it is generally preferred to proceed from virgin plant or animal raw materials in the production of these materials, substance mixtures having different alkyl chain lengths dependent on the particular raw material thus being obtained.

In the case of the surfactants that are products of the addition of ethylene oxide and/or propylene oxide to fatty alcohols or derivatives of these addition products, products having a “normal” homolog distribution as well as products having a restricted homolog distribution can be used. The term “normal homolog distribution” is understood to mean mixtures of homologs that are obtained when fatty alcohol and alkylene oxide are reacted by using alkali metals, alkali metal hydroxides, or alkali metal alcoholates as catalysts. In contrast, restricted homolog distributions are obtained if, for example, hydrotalcites, alkaline-earth metal salts of ether carboxylic acids, alkaline-earth metal oxides, alkaline-earth metal hydroxides, or alkaline-earth metal alcoholates are used as catalysts. The use of products having a restricted homolog distribution can be preferred.

Cleaning agents preferred according to the invention include at least one nonionic surfactant in a total amount of 0.01 to 10 wt %, preferably 0.1 to 8 wt %, especially preferably 0.2 to 6 wt %, extraordinarily preferably 0.5 to 5 wt %, further extraordinarily preferably 1 to 3 wt %, with respect to the weight of the cleaning agent. Care agents preferred according to the invention also include at least one nonionic surfactant, preferably in total amounts of 0.01 to 7 wt %, preferably 0.1 to 5 wt %, especially preferably 0.1 to 3 wt %, with respect to the weight of the care agent.

To achieve an optimal cleaning result, it is preferred that mixtures of different surfactant types are used. Preferred cleaning agents according to the invention include mixtures of at least one anionic surfactant and at least one zwitterionic surfactant. Other preferred cleaning agents according to the invention include mixtures of at least one anionic surfactant and at least one ampholytic surfactant. Other preferred cleaning agents according to the invention include mixtures of at least one anionic surfactant, at least one zwitterionic surfactant, and at least one nonionic surfactant.

In principle, all cationic surface-active substances suitable for use on the human body are suitable as cationic surfactants in cleaning and/or care agents according to the invention. These are characterized by at least one water-solubilizing cationic group, such as a quaternary ammonium group, or by at least one water-solubilizing cationizable group, such as an amine group, and also at least one lipophilic alkyl group having approximately 6 to 30 C atoms, or by at least one imidazole group or at least one imidazyl alkyl group.

Preferred cleaning and/or care agents, particularly care agents, according to the invention include at least one cationic surfactant, which is preferably selected from quaternary ammonium compounds having at least one C8-C24 alkyl residue, esterquats and amidoamines having at least one C8-C24 acyl residue, and mixtures thereof. Preferred quaternary ammonium compounds having at least one C8-C24 alkyl residue are ammonium halides, particularly chlorides, and ammonium alkyl sulfates, such as methosulfates or ethosulfates, such as C8-C24-alkyltrimethylammonium chlorides, C8-C24-dialkyldimethylammonium chlorides, and C8-C24-trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, and the imidazolium compounds known under the INCI names Quaternium-27, Quaternium-83, Quaternium-87, and Quaternium-91. The alkyl chains of the aforementioned surfactants have preferably 8 to 24 carbon atoms.

Esterquats are cationic substances that include both at least one ester function and at least one quaternary ammonium group as a structural element and also at least one C8-C24 alkyl residue or C8-C24 acyl residue. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are sold, for example, under the trademarks Stepantex®, Dehyquart®, and Armocare®. N,N-bis(2-palmitoyloxyethyl)dimethyl ammonium chloride, distearoylethyl dimonium methosulfate, and distearoylethyl hydroxyethylmonium methosulfate are preferred examples of such esterquats.

The alkylamidoamines are produced typically by the amidation of natural or synthetic C8-C24 fatty acids and fatty acid cuts with di-(C1-C3)-alkylaminoamines. A compound from this substance group that is especially suitable according to the invention is stearamidopropyl dimethylamine.

Care agents preferred according to the invention include at least one cationic surfactant in a total amount of 0.01 to 8 wt %, preferably 0.1 to 6 wt %, especially preferably 0.5 to 5 wt %, extraordinarily preferably 1.0 to 4 wt %, further extraordinarily preferably 1.5 to 3.5 wt %, with respect to the weight of the care agent. Cleaning agents preferred according to the invention also include at least one cationic surfactant, preferably in a total amount of 0.01 to 2 wt %, preferably 0.1 to 1 wt %, especially preferably 0.2 to 0.5 wt %, with respect to the weight of the cleaning agent.

Fatty Alcohols

Other cleaning and/or care agents, particularly care agents, preferred according to the invention are characterized in that at least one linear saturated 1-alkanol having 12 to 30 carbon atoms is included, preferably in a total amount of 0.2 to 10 wt %, especially preferably 0.5 to 7 wt %, with respect to the weight of the cleaning and/or care agent. These linear saturated 1-alkanols are also called fatty alcohols.

The at least one linear saturated 1-alkanol having 12 to 30 carbon atoms is preferably selected from lauryl alcohol, myristyl alcohol, cetyl alcohol, stearyl alcohol, arachidyl alcohol, and behenyl alcohol and from mixtures of these 1-alkanols, especially preferably from cetyl alcohol, stearyl alcohol, arachidyl alcohol, behenyl alcohol, and cetyl alcohol/stearyl alcohol mixtures.

Preferred cleaning and/or care agents according to the invention include, with respect to the weight thereof, at least one linear saturated 1-alkanol having 12 to 30 carbon atoms in a total amount of 0.2-10 wt %, preferably in a total amount of 0.5-7 wt %, wherein at least one 1-alkanol selected from cetyl alcohol, stearyl alcohol, behenyl alcohol, and cetyl alcohol/stearyl alcohol mixtures is included.

Oils

Natural and synthetic cosmetic oil bodies that are care active substances according to the invention are, in particular:
plant oils. Examples of preferred plant oils are sunflower oil, olive oil, soy oil, rape oil, almond oil, jojoba oil, orange oil, wheat germ oil, peach kernel oil, and the liquid fractions of coconut oil. Other triglyceride oils, such as the liquid fractions of beef tallow (animal) and synthetic triglyceride oils, particularly C8-C10 triglycerides, are also suitable.
liquid paraffin oils, isoparaffin oils, and synthetic hydrocarbons and di-n-alkyl ethers having a total of 12 to 36 C atoms, particularly 12 to 24 C atoms, such as di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether, and n-hexyl n-undecyl ether, and di-tert-butyl ether, diisopentyl ether, di-3-ethyldecyl ether, tert-butyl n-octyl ether, isopentyl n-octyl ether, and 2-methylpentyl n-octyl ether. The compounds 1,3-di(2-ethylhexyl)-cyclohexane (Cetiol® S) and di-n-octyl ether (Cetiol® OE) can be preferred.

Other cosmetic oils that are preferred according to the invention are selected from the silicone oils, which include, for example, dialkyl and alkylaryl siloxanes, such as cyclopentasiloxane, cyclohexasiloxane, dimethylpolysiloxane, and methylphenylpolysiloxane, and hexamethyldisiloxane, octamethyltrisiloxane, and decamethyltetrasiloxane. Volatile silicone oils, which can be cyclic, can be preferred, such as octamethylcyclotetrasiloxane, decamethylcyclopentasiloxane, and dodecamethylcyclohexasiloxane and mixtures thereof, which are included, for example, in the commercial products DC 244, 245, 344, and 345 from Dow Corning. Likewise suitable are volatile linear silicone oils, particularly hexamethyldisiloxane (L₂), octamethyltrisiloxane (L₃), decamethyltetrasiloxane (L₄), and any two-component and three-component mixtures of L₂, L₃, and/or L₄, preferably mixtures that are included, for example, in the commercial products DC 2-1184, Dow Corning® 200 (0.65 cSt), and Dow Corning® 200 (1.5 cSt) from Dow Corning. Preferred non-volatile silicone oils are selected from high-molecular-weight linear dimethylpolysiloxanes, which are commercially available, for example, under the name Dow Corning® 190, Dow Corning® 200 Fluid, having kinematic viscosities (25° C.) in the range of 5-100 cSt, preferably 5-50 cSt or even 5-10 cSt, and dimethylpolysiloxane having a kinematic viscosity (25° C.) of approximately 350 cSt.

It can be extraordinarily preferred according to the invention that mixtures of the aforementioned oils are used.

Preferred cleaning and/or care agents, particularly care agents, according to the invention include, with respect to the weight thereof, at least one oil in a total amount of 0.01 to 98 wt %, preferably 0.1 to 50 wt %, especially preferably 0.5 to 10 wt %, extraordinarily preferably 1 to 3 wt %, with respect to the weight of the cleaning and/or care agent.

Preferred cleaning and/or care agents according to the invention include water in an amount of 0.5 to 98 wt %, especially preferably 10 to 87 wt %, and extraordinarily preferably 45 to 80 wt %, with respect to the weight of the cleaning and/or care agent.

Shampoos

Preferred cleaning agents according to the invention are a shampoo and include, in addition to the at least one saturated dicarboxylic acid having 2 to 10 carbon atoms and the at least one amino acid according to formula (VI):
water in an amount of 45 to 98 wt %, especially preferably 60 to 93 wt %, and extraordinarily preferably 70 to 89 wt %, with respect to the weight of the cleaning agent,
at least one anionic surfactant in a total amount of 0.1 to 30 wt %, preferably 0.5 to 20 wt %, especially preferably 1 to 15 wt %, extraordinarily preferably 3 to 10 wt %, further extraordinarily preferably 5 to 7 wt %, with respect to the weight of the cleaning agent, wherein preferably at least one anionic surfactant selected from alkyl polyglycol ether sulfates and ether carboxylic acids having 10 to 18 C atoms in the alkyl group and 2 to 6 ethylene oxide groups in the molecule, acyl isethionates having 8 to 24 C atoms in the acyl group, and acylated amino acids and protein fatty acid condensates having 8 to 24 C atoms in the acyl group and from mixtures of these anionic surfactants is included, and
optionally at least one zwitterionic surfactant in a total amount of 0.1 to 15 wt %, preferably 0.2 to 10 wt %, especially preferably 0.5 to 8 wt %, extraordinarily preferably 1 to 5 wt %, further extraordinarily preferably 2 to 4 wt %, with respect to the weight of the cleaning agent, wherein cocamidopropyl betaine is especially preferably included.

Conditioning Compositions

Preferred care agents according to the invention are a rinse-off hair-conditioning composition and include, in addition to the at least one saturated dicarboxylic acid having 2 to 10 carbon atoms and the at least one amino acid according to formula (VI):
water in an amount of 45 to 98 wt %, especially preferably 60 to 93 wt %, and extraordinarily preferably 70 to 89 wt %, with respect to the weight of the cleaning agent,
at least one cationic surfactant in a total amount of 0.01 to 8 wt %, preferably 0.1 to 6 wt %, especially preferably 0.5 to 5 wt %, extraordinarily preferably 1.0 to 4 wt %, further extraordinarily preferably 1.5 to 3.5 wt %, with respect to the weight of the care agent, wherein preferably at least one cationic surfactant selected from quaternary ammonium compounds having at least one C8-C24 alkyl residue, esterquats and amidoamines having at least one C8-C24 acyl residue, and mixtures thereof is included,
at least one linear saturated 1-alkanol having 12 to 30 carbon atoms in a total amount of 0.2 to 10 wt %, especially preferably 0.5 to 7 wt %, with respect to the weight of the care agent, wherein at least one linear saturated 1-alkanol selected from lauryl alcohol, myristyl alcohol, cetyl alcohol, stearyl alcohol, arachidyl alcohol, and behenyl alcohol and from mixtures of these 1-alkanols is included, and
optionally at least one oil in a total amount of 0.01 to 10 wt %, preferably 0.1 to 3 wt %, especially preferably 0.5 to 1 wt %, with respect to the weight of the care agent.

Optional: Polymer A having at Least 10 Constitutive Units of Formula (I)

Cleaning and/or care agents preferred according to the invention optionally include at least one polymer A, which has at least ten constitutive units of formula (I),

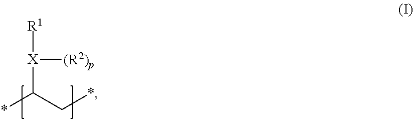

wherein
X represents nitrogen or oxygen and
R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
p=0 if X represents oxygen and p=1 if X represents nitrogen,
   wherein the polymer A does not include any permanently ionic constitutive units.

Surprisingly, it has been found that a polymer A, as described above and explained in more detail below, excellently supports the protective and reparative effect and the restructuring effect of the cleaning and/or care agents according to the invention that the combination of at least one saturated dicarboxylic acid having 2 to 10 carbon atoms and/or at least one salt of said acid(s) and at least one amino acid of formula (VI), as explained above, has on damaged keratin fibers.

In the sense of the present invention, the term “polymer” is understood to mean polymers in the sense of the IUPAC definition that comprise at least 10 identical constitutive units.

According to the RÖMPP Chemie Lexikon, July 2009 edition, a substance composed of a collective of chemically uniformly constructed macromolecules (polymer molecules) is referred to as a polymer in accordance with a definition of the IUPAC, wherein said macromolecules or polymer molecules differ from each other in regard to the degree of polymerization, molar mass, and chain length. Thus, in the case of such polymerically uniform materials, all macromolecules are identically constructed and differ merely in the chain length thereof (degree of polymerization). According to this IUPAC definition, a polymer is furthermore a polyreaction product that is constructed of a plurality of molecules, in which one type or several types of atoms or atom groupings (constitutive units, basic building blocks, or repeat units) are strung together repetitively.

The number of constitutive units in a polymer is called the degree of polymerization. Polymers A and polymers B preferred according to the invention have a degree of polymerization in the range of 40 to 1000, preferably 100 to 800, especially preferably 350 to 650. Other polymers A preferred according to the invention having at least ten constitutive units of formula (I) include 40 to 1000, preferably 100 to 800, especially preferably 350 to 650 identical constitutive units of formula (I).

R¹ and R² preferably represent, independently of each other, hydrogen or a C₂-C₁₀ acyl group, which preferably selected from an acetyl, propanoyl, or n-butanoyl group, especially preferably from an acetyl group.

Polymers A preferred according to the invention have at least 10 constitutive units of formula (I), in which X represents nitrogen, wherein the polymer A does not include any permanently ionic constitutive units.

Other polymers A especially preferred according to the invention have at least 10 constitutive units of formula (I), in which X represents nitrogen and R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group.

If R¹ and R² form a five- or six-membered, saturated or unsaturated ring together with X, which ring optionally includes further heteroatoms, which are preferably selected from N and O, said ring is preferably substituted with at least one functional group, which is selected from ═O. An especially preferred substituent combination X, R¹, R² is a pyrrolidone group, a constitutive unit of formula (I) especially preferred according to the invention therefore being a unit of formula (Ia),

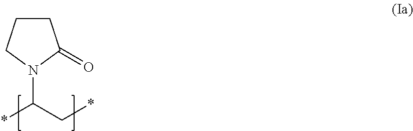

in which X represents nitrogen and R¹ and R² form a five-membered saturated ring together with this nitrogen atom, which ring does not include any further heteroatoms and is substituted with a functional group ═O in the 2 position.

Another especially preferred substituent combination X, R¹, R² is an ε-caprolactam group, a constitutive unit of formula (I) especially preferred according to the invention therefore being a unit of formula (I b),

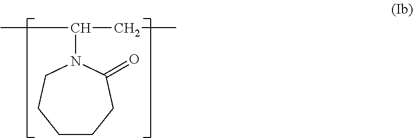

in which X represents nitrogen and R¹ and R² form a six-membered saturated ring together with this nitrogen atom, which ring does not include any further heteroatoms and is substituted with a functional group ═O.

Another especially preferred substituent combination X, R¹, R² is an imidazole group, another unit of formula (I) especially preferred according to the invention therefore being a unit in which X represents nitrogen and R¹ and R² form a five-membered unsaturated ring together with this nitrogen atom, which ring includes nitrogen as a further heteroatom.

Other polymers A preferred according to the invention have 25-100 mol %, preferably 55-100 mol %, especially preferably 85-100 mol % of constitutive units of formula (I), in which X represents nitrogen, wherein the polymer A does not include any permanently ionic constitutive units.

Other polymers A preferred according to the invention have 25-100 mol %, preferably 55-100 mol %, especially preferably 85-100 mol % of constitutive units of formula (I), in which X represents nitrogen and R¹ and R² form a five- or six-membered, saturated or unsaturated ring together with X, which ring optionally includes further heteroatoms, which are preferably selected from N and O, and is optionally substituted with at least one C1-C6 alkyl group and/or with at least one functional group, wherein the polymer A does not include any permanently ionic constitutive units.

Polymers A especially preferred according to the invention have 98-100 mol % of constitutive units of formula (Ia), wherein the polymer A does not include any permanently ionic constitutive units.

Polymers A extraordinarily preferred according to the invention have 98-100 mol % of constitutive units of formula (Ia) and have a degree of polymerization in the range of 40 to 1000, preferably 100 to 800, especially preferably 350 to 650, wherein the polymer A does not include any permanently ionic constitutive units. Especially preferred polymers A are polyvinylpyrrolidone homopolymers having a degree of polymerization in the range of 40 to 1000, preferably 100 to 800, especially preferably 350 to 650.

Another especially preferred substituent combination X, R¹, R² is a constitutive unit of formula (I), in which X represents oxygen, p is zero, and R¹ represents hydrogen.

Another especially preferred substituent combination X, R¹, R² is a constitutive unit of formula (I), in which X represents oxygen, p is zero, and R¹ represents an acetyl group.

Other polymers A preferred according to the invention include 75-92 mol % of constitutive units of formula (I), in which X represents oxygen, p is zero, and R¹ represents hydrogen, and 8-25 mol % of constitutive units of formula (I), in which X represents oxygen, p is zero, and R¹ represents an acetyl group, wherein the polymer A does not include any permanently ionic constitutive units.

Other polymers A preferred according to the invention include 40 to 1000, preferably 100 to 800, especially preferably 350 to 650 constitutive units of formula (I), thereof 75-92 mol % of constitutive units of formula (I), in which X represents oxygen, p is zero, and R¹ represents hydrogen, and 8-25 mol % of constitutive units of formula (I), in which X represents oxygen, p is zero, and R¹ represents an acetyl group, wherein the polymer A does not include any permanently ionic constitutive units.

Other polymers A preferred according to the invention include 65-25 mol % of constitutive units of formula (Ia) and 35-75 mol % of constitutive units of formula (I), in which X represents oxygen, p is zero, and R¹ represents an acetyl group, wherein the polymer A does not include any permanently ionic constitutive units.

Other polymers A preferred according to the invention include 40 to 1000, preferably 100 to 800, especially preferably 350 to 650 constitutive units of formula (I), thereof 65-25 mol % of constitutive units of formula (Ia) and 35-75 mol % of constitutive units of formula (I), in which X represents oxygen, p is zero, and R¹ represents an acetyl group, wherein the polymer A does not include any permanently ionic constitutive units.

The at least one polymer A having at least ten constitutive units of formula (I) does not have any permanent ionic charges. However, it is possible that the constitutive units of formula (I) exist in ionic form, particularly cationic form, for example as the result of the protonation of the nitrogen atom in an acidic carrier. But these charges are not permanent, but rather temporary, because these charges depend on the surrounding medium.

Preferred cleaning and/or care agents according to the invention include the at least one polymer A having at least ten constitutive units of formula (I) in a total amount of 0.2 to 5 wt %, preferably 0.5 to 3 wt %, especially preferably 1.0 to 2.3 wt %, with respect to the weight of the cleaning and/or care agent.

Other preferred cleaning and/or care agents according to the invention include at least one polymer A having at least ten constitutive units of formula (I), selected from polyvinylpyrrolidone, polyvinyl alcohol, and mixtures thereof, wherein polyvinylpyrrolidone is especially preferred.

Other preferred cleaning and/or care agents according to the invention include at least one polymer A having at least ten constitutive units of formula (I) in a total amount of 0.2 to 5 wt %, preferably 0.5 to 3 wt %, especially preferably 1.0 to 2.3 wt %, with respect to the weight of the cleaning and/or care agent, wherein the polymer A is selected from polyvinylpyrrolidone, polyvinyl alcohol, and mixtures thereof, wherein polyvinylpyrrolidone is especially preferred.

Furthermore, it was surprisingly found that the protective and reparative effect and the restructuring effect of the cleaning and/or care agents according to the invention and preferred according to the invention that the combination of at least one saturated dicarboxylic acid having 2 to 10 carbon atoms and/or at least one salt of said acid(s) and at least one amino acid of formula (VI), as explained above, has on damaged keratin fibers can be further supported if at least one compound of general formula (III) is included.

Therefore, cleaning and/or care agents preferred according to the invention include
(a) at least one compound of general formula (III)

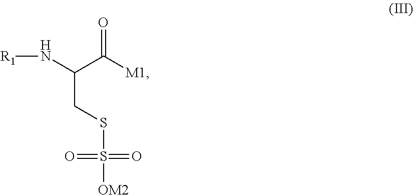

wherein
R1 represents a hydrogen atom or a structural element of formula (IV),

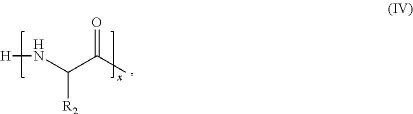

wherein
x represents an integer from 1 to 100,
the residue R2 in each of the structural elements of formula (IV) can be selected independently of the preceding structural element of formula (IV),
R2 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group,
M1 represents the group —OM2 or a structural element of formula (V),

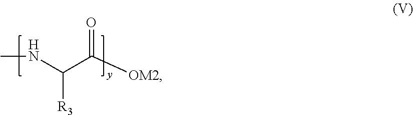

wherein
y represents an integer from 1 to 100,
the residue R3 in each of the structural elements of formula (V) can be selected independently of the preceding structural element of formula (V),
R3 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group,
M2 represents a hydrogen atom, an equivalent of a mono- or polyvalent cation, or an ammonium ion (NH₄)⁺.

The essential ingredient (a) of formula (III) is the Bunte salt of an amino acid, of an oligopeptide, or of a peptide, said Bunte salt being a compound of formula (III),

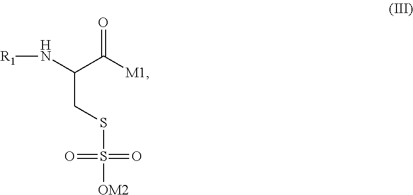

wherein
R1 represents a hydrogen atom or a structural element of formula (IV),

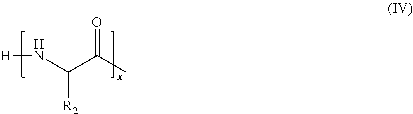

wherein
x represents an integer from 1 to 100,
the residue R2 in each of the structural elements of formula (IV) can be selected independently of the preceding structural element of formula (IV),
R2 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group,
M1 represents the group —OM2 or a structural element of formula (V),

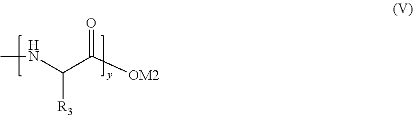

wherein
y represents an integer from 1 to 100,
the residue R3 in each of the structural elements of formula (V) can be selected independently of the preceding structural element of formula (V),
R3 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group,
M2 represents a hydrogen atom, an equivalent of a mono- or polyvalent cation, or an ammonium ion (NH₄)⁺.

The residue R1 can represent either a hydrogen atom or a structural element of formula (IV),

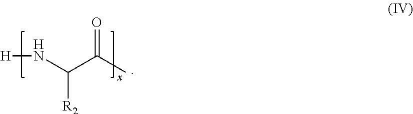

Furthermore, the structural element of formula (IV) is characterized by the repetition index x, wherein x represents an integer from 1 to 100. The repetition index x indicates how many structural elements of formula (IV) are included in the compound of formula (III).

x preferably represents an integer from 1 to 50, x more preferably represents an integer from 1 to 20, and x exceedingly preferably represents an integer from 1 to 10.

If x represents the number 10, for example, the compound of formula (III) includes 10 structural elements of formula (IV).

It is essential here that the residue R2 in each of the structural elements of formula (IV) can be selected independently of the preceding structural element of formula (IV). If the compounds of formula (III) include, for example, 10 structural units of formula (IV), said 10 structural units can be the same or different.

The residue R2 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group.

Therefore, the structural element of formula (IV) is an amino acid that is peptidically linked by means of the amino function thereof and/or the acid function thereof within the compound of formula (III). If the amino acid is cysteine, the amino acid can also exist in the form of a Bunte salt.

If the residue R2 represents a hydrogen atom, the structural element of formula (IV) is based on the amino acid glycine.

If the residue R2 represents a methyl group, the structural element of formula (IV) is based on the amino acid alanine.

If the residue R2 represents an isopropyl group (i.e., a group (H₃C)₂CH—), the structural element of formula (IV) is based on the amino acid valine.

If the residue R2 represents a 2-methylpropyl group (i.e., a group (H₃C)₂CH—CH₂—), the structural element of formula (IV) is based on the amino acid leucine.

If the residue R2 represents a 1-methylpropyl group (i.e., a group H3C—CH2-CH(CH3)—), the structural element of formula (IV) is based on the amino acid isoleucine.

If the residue R2 represents a benzyl group (i.e., a group C₆H₅—CH₂—), the structural element of formula (IV) is based on the amino acid phenylalanine.

If the residue R2 represents a 4-hydroxybenzyl group (i.e., a group 4-OH—C₆H₅—CH₂—), the structural element of formula (IV) is based on the amino acid tyrosine.

If the residue R2 represents a hydroxymethyl group (i.e., a group HO—CH2-), the structural element of formula (IV) is based on the amino acid serine.

If the residue R2 represents a 1-hydroxyethyl group (i.e., a group H3C—CH(OH)—), the structural element of formula (IV) is based on the amino acid threonine.

If the residue R2 represents a 4-aminobutyl group (i.e., a group H2N—CH2-CH2-CH2-CH2-), the structural element of formula (IV) is based on the amino acid lysine.

If the residue R2 represents a 3-carbamimidamidopropyl group (i.e., a group H₂N—C(NH)—NH—CH₂—CH₂—CH₂—), the structural element of formula (IV) is based on the amino acid arginine.

If the residue R2 represents a 2-carboxyethyl group (i.e., a group HOOC—CH2-CH2-), the structural element of formula (IV) is based on the amino acid glutamic acid.

If the residue R2 represents a carboxymethyl group (i.e., a group HOOC—CH2-), the structural element of formula (IV) is based on the amino acid aspartic acid.

If the residue R2 represents a 2-carbamoylethyl group (i.e., a group H2N—C(O)—CH2-CH2-), the structural element of formula (IV) is based on the amino acid glutamine.

If the residue R2 represents a carbamoylmethyl group (i.e., a group H2N—C(O)—CH2-), the structural element of formula (IV) is based on the amino acid asparagine.

If the residue R2 represents a sulfanylmethyl group (i.e., a group HS—CH2-), the structural element of formula (IV) is based on the amino acid cysteine.

If the residue R2 represents a 2-(methylsulfanyl)ethyl group (i.e., a group H3C—S—CH2-CH2-), the structural element of formula (IV) is based on the amino acid methionine.

If the residue R2 represents a 1H-imidazol-4-ylmethyl group, the structural element of formula (IV) is based on the amino acid histidine.

If the residue R2 represents a 1H-indol-3-ylmethyl group, the structural element of formula (IV) is based on the amino acid tryptophan.

Finally, the residue R2 can also represent a (sulfosulfanyl)methyl group, resulting in a Bunte salt structure of the formula HO—S(O₂)—S—CH₂—.

The Bunte salt structure of the formula HO—S(O₂)—S—CH₂— can also exist in the deprotonated form thereof, depending on the pH value of the cleaning and/or care agent according to the invention or preferred according to the invention or of the composition (A) used according to the invention or preferably used according to the invention.

Within the compound of formula (III), M1 represents the group —OM2 or a structural element of formula (V),

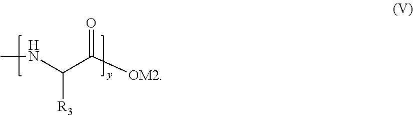

The structural element of formula (V)—exactly like the structural element of formula (IV)—is characterized by the repetition index y, wherein y represents an integer from 1 to 100. The repetition index y indicates how many structural elements of formula (V) are included in the compound of formula (III).

y preferably represents an integer from 1 to 50, y more preferably represents an integer from 1 to 20, and y exceedingly preferably represents an integer from 1 to 10.

If y represents the number 10, for example, the compound of formula (III) includes 10 structural elements of formula (V).

It is essential here that the residue R3 in each of the structural elements of formula (V) can be selected independently of the preceding structural element of formula (V). If the compounds of formula (III) include, for example, 10 structural units of formula (V), said 10 structural units can be the same or different.

The residue R3 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group.

Therefore, the structural element of formula (V) also is an amino acid that is peptidically linked by means of the amino function thereof and/or the acid function thereof within the compound of formula (III). If the amino acid is cysteine, the amino acid can also exist in the form of a Bunte salt.

If the residue R3 represents a hydrogen atom, the structural element of formula (IV) is based on the amino acid glycine.

If the residue R3 represents a methyl group, the structural element of formula (IV) is based on the amino acid alanine.

If the residue R3 represents an isopropyl group (i.e., a group (H₃C)₂CH—), the structural element of formula (IV) is based on the amino acid valine.

If the residue R3 represents a 2-methylpropyl group (i.e., a group (H₃C)₂CH—CH₂—), the structural element of formula (IV) is based on the amino acid leucine.

If the residue R3 represents a 1-methylpropyl group (i.e., a group H3C—CH2-CH(CH3)-), the structural element of formula (IV) is based on the amino acid isoleucine.

If the residue R3 represents a benzyl group (i.e., a group C₆H₅—CH₂—), the structural element of formula (IV) is based on the amino acid phenylalanine.

If the residue R3 represents a 4-hydroxybenzyl group (i.e., a group 40H—C₆H₅—CH₂—), the structural element of formula (IV) is based on the amino acid tyrosine.

If the residue R3 represents a hydroxymethyl group (i.e., a group HO—CH2-), the structural element of formula (IV) is based on the amino acid serine.

If the residue R3 represents a 1-hydroxyethyl group (i.e., a group H3C—CH(OH)—), the structural element of formula (IV) is based on the amino acid threonine.

If the residue R3 represents a 4-aminobutyl group (i.e., a group H2N—CH₂-CH2-CH2-CH2-), the structural element of formula (IV) is based on the amino acid lysine.

If the residue R3 represents a 3-carbamimidamidopropyl group (i.e., a group H₂N—C(NH)—NH—CH₂—CH₂—CH₂—), the structural element of formula (IV) is based on the amino acid arginine.

If the residue R3 represents a 2-carboxyethyl group (i.e., a group HOOC—CH2-CH2-), the structural element of formula (IV) is based on the amino acid glutamic acid.

If the residue R3 represents a carboxymethyl group (i.e., a group HOOC—CH2-), the structural element of formula (IV) is based on the amino acid aspartic acid.

If the residue R3 represents a 2-carbamoylethyl group (i.e., a group H2N—C(O)—CH2-CH2-), the structural element of formula (IV) is based on the amino acid glutamine.

If the residue R3 represents a carbamoylmethyl group (i.e., a group H2N—C(O)—CH2-), the structural element of formula (IV) is based on the amino acid asparagine.

If the residue R3 represents a sulfanylmethyl group (i.e., a group HS—CH2-), the structural element of formula (IV) is based on the amino acid cysteine.

If the residue R3 represents a 2-(methylsulfanyl)ethyl group (i.e., a group H3C—S—CH2-CH2-), the structural element of formula (IV) is based on the amino acid methionine.

If the residue R3 represents a 1H-imidazol-4-ylmethyl group, the structural element of formula (IV) is based on the amino acid histidine.

If the residue R3 represents a 1H-indol-3-ylmethyl group, the structural element of formula (IV) is based on the amino acid tryptophan.

Finally, the residue R3 can also represent a (sulfosulfanyl)methyl group, resulting in a Bunte salt structure of the formula HO—S(O₂)—S—CH2-.

Here as well, the Bunte salt structure of the formula HO—S(O₂)—S—CH₂— can also exist in the deprotonated form thereof, depending on the pH value of the cleaning and/or care agent according to the invention or preferred according to the invention or of the composition (A) used according to the invention or preferably used according to the invention.

The residue M2 represents a hydrogen atom, an equivalent of a mono- or polyvalent cation, or an ammonium ion (NH₄)⁺.

In particular, the cations of sodium and potassium (Na⁺ and K⁺) or magnesium or calcium (½ Mg²⁺ or ½ Ca²⁺) can be mentioned as preferred equivalents of a mono- or polyvalent cation.

If M2 represents a hydrogen atom, the group —OM2 is the group —OH. If M2 represents a sodium cation, the group —OM2 is the group —ONa. If M2 represents a potassium cation, the group —OM2 is the group —OK. If M2 represents an ammonium ion, the group —OM2 is the group —O(NH₄)⁺.

The group —OM2 is always adjacent to a carbonyl group. In summation, if M2 represents H, K, Na, ammonium, either an acid in the protonated form thereof or the sodium, potassium, or ammonium salt of said acid is therefore present in the compound of formula (III).

The compounds of formula (III) according to the invention are either the Bunte salts of the amino acid cysteine, the Bunte salts of oligopeptides, or the Bunte salts of peptides.

If the residue R1 represents a hydrogen atom and the residue M1 represents a group —OM2, the compound of formula (III) is the Bunte salt of the amino acid cysteine. In this case, the compound of formula (III) is the compound of formula (IIIa),

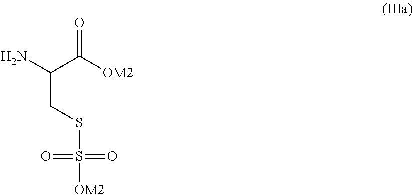

wherein M2 defined again as described above.

If the compound of formula (IIIa) exists in the form of the free acid thereof, the compound is 2-amino-3-(sulfosulfanyl)propanoic acid. This substance is commercially available.

It has been found that the use of the compound of formula (IIIa) in cleaning and/or care agents, even in especially low usage amounts, leads to an especially effective reduction in hair damage that is still present even after repeated hair washing. Therefore, the use of compounds of formula (IIIa) in cleaning and/or care agents is exceedingly preferred.

In an exceedingly preferred embodiment, a cleaning and/or care agent according to the invention is characterized in that said cleaning and/or care agent includes at least one compound of formula (III), wherein
R1 represents a hydrogen atom and
M1 represents a group —OM2.

If a compound of formula (IIIa) is used, then this specific compound is preferably used. If, however, the Bunte salts of oligopeptides are used as compounds of formula (III), then the cleaning and/or care agent according to the invention can also include several compounds of formula (III) as a mixture of different oligopeptides. Said oligopeptides are defined by means of the average molecular weight thereof. The average molecular weight M_{w} of the at least one oligopeptide of formula (III) can be determined, for example, by gel permeation chromatography (GPC) with polystyrene as an internal standard in accordance with DIN 55672-3, version 8/2007.

The molecular weight of the compound of formula (III) used according to the invention can vary depending on how many structural elements of formula (III) and/or (IV) are included in the compound of formula (III) and depending on the type of these amino acids. It is especially preferred according to the invention if the compound of formula (III) is an oligopeptide that has a molecular weight M_{w} of 200 to 2,000 Da, preferably 250 to 1,500 Da, preferably 300 to 1,200 Da, particularly 400 to 800 Da.

In the context of the present invention, the term “oligopeptide” is understood to mean condensation products of amino acids that have the aforementioned molecular weights.

In an exceedingly preferred embodiment, a cleaning and/or care agent according to the invention is characterized in that said cleaning and/or care agent includes at least one compound of formula (III), which has a molecular weight M_{w} of 200 to 2,000 Da (daltons), preferably 250 to 1,500 Da, preferably 300 to 1,200 Da, particularly 400 to 800 Da.

If a mixture of oligomers is used in the cleaning and/or care agent according to the invention, these mixtures can be defined by means of the average molecular weight thereof.

In this case, a preferred cleaning and/or care agent according to the invention is characterized in that said cleaning and/or care agent includes at least one mixture of compounds of formula (III), which mixture has an average molecular weight M_{w} of 200 to 2,000 Da, preferably 250 to 1,500 Da, preferably 300 to 1,200 Da, particularly 400 to 800 Da.

Furthermore, it has been found that the protective or reparative effect that the compounds of formula (III) have depends also on the repetition indices x and y. As previously described, it is exceedingly preferred if x represents an integer from 1 to 10 and y represents an integer from 1 to 10.

In another exceedingly preferred embodiment, a cleaning and/or care agent according to the invention is characterized in that said cleaning and/or care agent includes at least one compound of formula (III), wherein
R1 represents a structural element of formula (IV), and
M1 represents a structural element of formula (V), and
x represents an integer from 1 to 10, and
y represents an integer from 1 to 10.

In addition to the molecular weight of the compound of formula (III), the fraction of the Bunte salt units that are included in the compound of formula (III) also has a decisive influence on the effectiveness of the protective effect or repair effect of the compounds.

Compounds having at least one Bunte salt unit—as is present in the compound of formula (IIIa), for example—are very effective, particularly if they are used as a monomeric compound. Oligopeptides having at least one Bunte salt unit are especially effective if said oligopeptides have a low molecular weight of up to 1200, particularly 800 daltons.

If oligopeptides are used, it is, however, exceedingly advantageous if the compound of formula (III) has at least two, preferably at least three Bunte salt units.

In another exceedingly preferred embodiment, a cleaning and/or care agent according to the invention is characterized in that said cleaning and/or care agent includes at least one compound of formula (III), wherein
R1 represents a structural element of formula (IV), and
the residue R2 in at least one structural element of formula (IV) represents a (sulfosulfanyl)methyl group (i.e., a group HO—S(O₂)—S—CH₂—).

In another exceedingly preferred embodiment, a cleaning and/or care agent according to the invention is characterized in that said cleaning and/or care agent includes at least one compound of formula (III), wherein
R1 represents a structural element of formula (IV), and
x represents an integer of at least 3, and
the residue R2 in at least 3 structural elements of formula (IV) represents a 2-carboxyethyl group (i.e., a group HOOC—CH2-CH2-).

In another exceedingly preferred embodiment, a cleaning and/or care agent according to the invention is characterized in that said cleaning and/or care agent includes at least one compound of formula (III), wherein
M1 represents a structural element of formula (V), and
y represents an integer of at least 3, and
the residue R3 in at least 3 structural elements of formula (IV) represents a group (Glu).

The at least one compound of formula (III) is included in a total amount of 0.001 to 5 wt % with respect to the total weight of the cleaning and/or care agent preferred according to the invention. However, it has surprisingly been found that the compound(s) of formula (III) can bring about a very good reduction in hair damage even at low usage concentrations. This advantageous particularly if the at least one compound of formula (III) should be added to the cleaning and/or care agent according to the invention as an additive (for example, in the form of a care solution or repair solution) before said cleaning and/or care agent is applied to the hair. For this reason, it is especially advantageous if the cleaning and/or care agent preferred according to the invention includes one or more compounds of formula (III) stated above in a total amount of 0.001 to 2.5 wt %, more preferably 0.005 to 0.2 wt %, and especially preferably 0.01 to 0.07 wt %, with respect to the weight of the cleaning and/or care agent according to the invention.

In another exceedingly preferred embodiment, a cleaning and/or care agent according to the invention is characterized in that said cleaning and/or care agent includes one or more compounds of formula (III) stated above in a total amount of 0.001 to 2.5 wt %, more preferably 0.005 to 0.2 wt %, and especially preferably 0.01 to 0.07 wt %, with respect to the weight of the cleaning and/or care agent according to the invention.

In another exceedingly preferred embodiment, a cleaning and/or care agent according to the invention is characterized in that said cleaning and/or care agent has a pH value in the range of 3.0 to 7.5, preferably 3.5 to 6.5, especially preferably 4.0 to 5.5, measured at 20° C.

The present invention also relates to a method for cleaning and/or caring for keratin fibers, particularly human hair, wherein a cleaning or care agent according to the invention or preferred according to the invention, as described above, is applied to the keratin fibers, particularly to the human hair, and optionally rinsed out after an exposure time of 0.1 to 60 minutes, preferably 0.5 to 15 minutes, especially preferably 1 to 10 minutes.

The present invention also relates to a method for cleaning and/or caring for keratin fibers, particularly human hair, comprising the following steps:
I. providing a composition (A), including
   at least one saturated dicarboxylic acid having 2 to 10 carbon atoms in a total amount of 2 to 20 wt %, preferably 5 to 15 wt %, especially preferably 8 to 12 wt %, converted to the undissociated acid and with respect to the weight of the composition (A), wherein the dicarboxylic acid is preferably selected from succinic acid, malic acid, oxalic acid, malonic acid, adipic acid, pimelic acid, suberic acid, azelaic acid, sebacic acid, and mixtures of these acids, wherein succinic acid and malic acid are especially preferred and succinic is extraordinarily preferred,
   at least one amino acid of formula (VI)

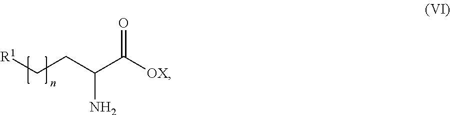

wherein
   X represents a hydrogen atom or a mono- or divalent cation;
   n represents 0, 1, 2, or 3;
   R¹ represents a residue that is selected from an amino group, a guanidine group, a (1H-imidazol-4-yl) group, a carboxylic acid amide group —CONH₂, a 1H-indol-3-yl group, a thiol group —SH, and a methylsulfanyl group —SCH₃, or at least one salt of said amino acid,
and/or one salt thereof in a total amount of 0.4 to 7.0 wt %, preferably 0.8 to 5.0 wt %, especially preferably 1.5 to 4.0 wt %, converted to the undissociated acid and with respect to the weight of the composition (A), wherein preferably at least one of the amino acids arginine, histidine, or lysine and/or one salt thereof is included in a total amount of 0.4 to 7.0 wt %, preferably 0.8 to 5.0 wt %, especially preferably 1.5 to 4.0 wt %, converted to the undissociated acid and with respect to the weight of the composition (A), and
   water, preferably in an amount of 50 to 92 wt %, especially preferably 60 to 87 wt %, and extraordinarily preferably 65 to 80 wt %, with respect to the weight of the composition (A),
   optionally also at least one polymer A, which has at least ten constitutive units of formula (I),

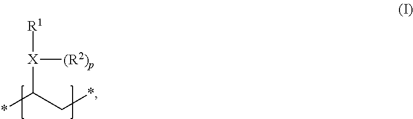

wherein
   X represents nitrogen or oxygen and
   R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
   p=0 if X represents oxygen and p=1 if X represents nitrogen,
wherein the polymer A does not include any permanently ionic constitutive units, wherein the at least one polymer A is preferably included in a total amount of 0.5 to 14 wt %, preferably 1.0 to 11 wt %, especially preferably 2.0 to 10 wt %, with respect to the weight of the composition (A),
wherein the composition (A) preferably has a pH value in the range of 3.5 to 7.1, preferably 4.5 to 6.5, especially preferably 5.0 to 6.0, measured at 20° C.,
   water, and
   optionally also at least one substance selected from
   compounds of general formula (III),

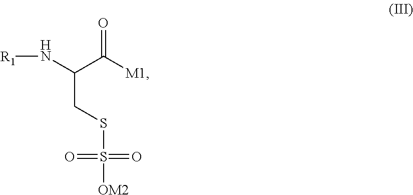

wherein
   R1 represents a hydrogen atom or a structural element of formula (IV),

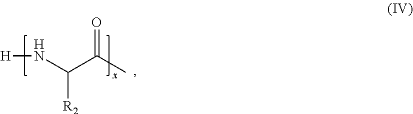

wherein
x represents an integer from 1 to 100,
the residue R2 in each of the structural elements of formula (IV) can be selected independently of the preceding structural element of formula (IV),
R2 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group,
M1 represents the group —OM2 or a structural element of formula (V),

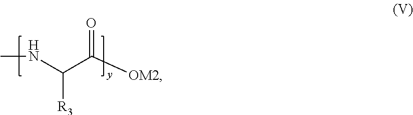

wherein
y represents an integer from 1 to 100,
the residue R3 in each of the structural elements of formula (V) can be selected independently of the preceding structural element of formula (V),
R3 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group,
M2 represents a hydrogen atom, an equivalent of a mono- or polyvalent cation, or an ammonium ion (NH₄)⁺,
wherein one or more compounds of formula (III) stated above are preferably included in a total amount of 0.01 to 2.5 wt %, more preferably 0.05 to 0.5 wt %, and especially preferably 0.1 to 0.3 wt %, with respect to the weight of the composition (A),
and
   polymers A, which have at least ten constitutive units of formula (I),

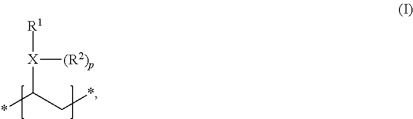

wherein
   X represents nitrogen or oxygen and
   R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
   p=0 if X represents oxygen and p=1 if X represents nitrogen,
wherein the polymer A does not include any permanently ionic constitutive units,
wherein the at least one polymer A is preferably included in a total amount of 0.5 to 14 wt %, preferably 1.0 to 11 wt %, especially preferably 2.0 to 10 wt %, with respect to the weight of the composition (A),
II. providing a cleaning and/or care composition (B), including
   at least one cleaning and/or care active substance, selected from surfactants, fatty alcohols, and oils,
   optionally water, and
III. mixing the compositions (A) and (B) with each other, and, directly thereafter,
IV. applying the mixture of (A) and (B) to the keratin fibers, particularly the human hair,
V. optionally rinsing out after an exposure time of 0.1 to 60 minutes, preferably 0.5 to 15 minutes, especially preferably 1 to 10 minutes,
VI. optionally performing further hair treatments, such as conditioning and/or drying.

The statements made above in respect of the cleaning and/or care agents according to the invention apply, mututatis mutandis, to the cleaning and/or care composition (B) with regard to water content, surfactant content, content of fatty alcohols, and content of oils.

The present invention also relates to a method for cleaning and/or caring for keratin fibers, wherein the composition (A) for treating hair includes:
at least one saturated dicarboxylic acid having 2 to 10 carbon atoms in a total amount of 2 to 20 wt %, preferably 5 to 15 wt %, especially preferably 8 to 12 wt %, converted to the undissociated acid and with respect to the weight of the composition (A), wherein the dicarboxylic acid is preferably selected from succinic acid, malic acid, oxalic acid, malonic acid, adipic acid, pimelic acid, suberic acid, azelaic acid, sebacic acid, and mixtures of these acids,
at least one amino acid of formula (VI) and/or one salt thereof in a total amount of 0.4 to 7.0 wt %, preferably 0.8 to 5.0 wt %, especially preferably 1.5 to 4.0 wt %, converted to the undissociated acid and with respect to the weight of the composition (A), and
water, preferably in an amount of 50 to 92 wt %, especially preferably 60 to 87 wt %, and extraordinarily preferably 65 to 80 wt %, with respect to the weight of the composition (A).

The present invention also relates to a method for cleaning and/or caring for keratin fibers, wherein the composition (A) for treating hair includes:
at least one saturated dicarboxylic acid having 2 to 10 carbon atoms in a total amount of 2 to 20 wt %, preferably 5 to 15 wt %, especially preferably 8 to 12 wt %, converted to the undissociated acid and with respect to the weight of the composition (A), wherein the dicarboxylic acid is preferably selected from succinic acid, malic acid, oxalic acid, malonic acid, adipic acid, pimelic acid, suberic acid, azelaic acid, sebacic acid, and mixtures of these acids,
at least one of the amino acids arginine, histidine, or lysine and/or one salt thereof in a total amount of 0.4 to 7.0 wt %, preferably 0.8 to 5.0 wt %, especially preferably 1.5 to 4.0 wt %, converted to the undissociated acid and with respect to the weight of the composition (A), and
50 to 92 wt %, especially preferably 60 to 87 wt %, and extraordinarily preferably 65 to 80 wt % of water, with respect to the weight of the composition (A).

The present invention also relates to a method for cleaning and/or caring for keratin fibers, wherein the composition (A) for treating hair includes:
at least one saturated dicarboxylic acid having 2 to 10 carbon atoms in a total amount of 2 to 20 wt %, preferably 5 to 15 wt %, especially preferably 8 to 12 wt %, converted to the undissociated acid and with respect to the weight of the composition (A), wherein the dicarboxylic acid is preferably selected from succinic acid, malic acid, oxalic acid, malonic acid, adipic acid, pimelic acid, suberic acid, azelaic acid, sebacic acid, and mixtures of these acids,
at least one amino acid of formula (VI) and/or one salt thereof in a total amount of 0.4 to 7.0 wt %, preferably 0.8 to 5.0 wt %, especially preferably 1.5 to 4.0 wt %, converted to the undissociated acid and with respect to the weight of the composition (A),
water, preferably in an amount of 50 to 92 wt %, especially preferably 60 to 87 wt %, and extraordinarily preferably 65 to 80 wt %, with respect to the weight of the composition (A), and
at least one polymer A, which has at least ten constitutive units of formula (I),

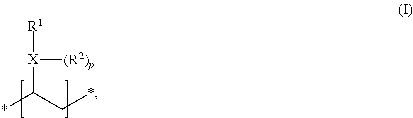

wherein
   X represents nitrogen or oxygen and
   R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
   p=0 if X represents oxygen and p=1 if X represents nitrogen,
wherein the polymer A does not include any permanently ionic constitutive units, in the composition (A) in a total amount of 0.5 to 14 wt %, preferably 1.0 to 11 wt %, especially preferably 2.0 to 10 wt %, with respect to the weight of the composition (A).

The present invention also relates to a method for cleaning and/or caring for keratin fibers, wherein the composition (A) for treating hair includes:
succinic acid in an amount of 2 to 20 wt %, preferably 5 to 15 wt %, especially preferably 8 to 12 wt %, converted to the undissociated acid and with respect to the weight of the composition (A),
at least one of the amino acids arginine, histidine, or lysine and/or one salt thereof in a total amount of 0.4 to 7.0 wt %, preferably 0.8 to 5.0 wt %, especially preferably 1.5 to 4.0 wt %, converted to the undissociated acid and with respect to the weight of the composition (A),
50 to 92 wt %, especially preferably 60 to 87 wt %, and extraordinarily preferably 65 to 80 wt % of water, with respect to the weight of the composition (A), and
at least one polymer A, which has at least ten constitutive units of formula (I),

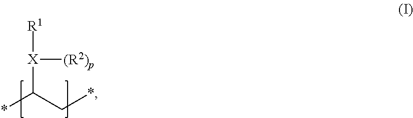

wherein
   X represents nitrogen or oxygen and
   R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
   p=0 if X represents oxygen and p=1 if X represents nitrogen,
wherein the polymer A does not include any permanently ionic constitutive units, in the composition (A) in a total amount of 0.5 to 14 wt %, preferably 1.0 to 11 wt %, especially preferably 2.0 to 10 wt %, with respect to the weight of the composition (A).

The present invention also relates to a method for cleaning and/or caring for keratin fibers, wherein the composition (A) for treating hair includes:
malic acid in an amount of 2 to 20 wt %, preferably 5 to 15 wt %, especially preferably 8 to 12 wt %, converted to the undissociated acid and with respect to the weight of the composition (A),
at least one of the amino acids arginine, histidine, or lysine and/or one salt thereof in a total amount of 0.4 to 7.0 wt %, preferably 0.8 to 5.0 wt %, especially preferably 1.5 to 4.0 wt %, converted to the undissociated acid and with respect to the weight of the composition (A),
50 to 92 wt %, especially preferably 60 to 87 wt %, and extraordinarily preferably 65 to 80 wt % of water, with respect to the weight of the composition (A), and
at least one polymer A, which has at least ten constitutive units of formula (I),

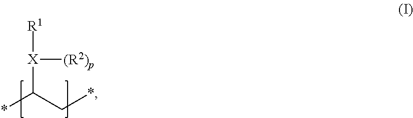

wherein
   X represents nitrogen or oxygen and
   R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
   p=0 if X represents oxygen and p=1 if X represents nitrogen,
wherein the polymer A does not include any permanently ionic constitutive units, in the composition (A) in a total amount of 0.5 to 14 wt %, preferably 1.0 to 11 wt %, especially preferably 2.0 to 10 wt %, with respect to the weight of the composition (A).

The present invention also relates to a method for cleaning and/or caring for keratin fibers, wherein the composition (A) for treating hair includes:
succinic acid in an amount of 2 to 20 wt %, preferably 5 to 15 wt %, especially preferably 8 to 12 wt %, converted to the undissociated acid and with respect to the weight of the composition (A),
at least one of the amino acids arginine, histidine, or lysine and/or one salt thereof in a total amount of 0.4 to 7.0 wt %, preferably 0.8 to 5.0 wt %, especially preferably 1.5 to 4.0 wt %, converted to the undissociated acid and with respect to the weight of the composition (A),
50 to 92 wt %, especially preferably 60 to 87 wt %, and extraordinarily preferably 65 to 80 wt % of water, with respect to the weight of the composition (A), and
at least one polymer A, which has at least ten constitutive units of formula (I),

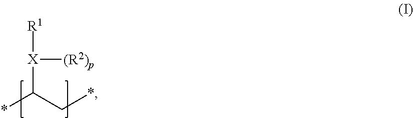

wherein
   X represents nitrogen or oxygen and
   R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
   p=0 if X represents oxygen and p=1 if X represents nitrogen,
wherein the polymer A does not include any permanently ionic constitutive units and is selected from polyvinylpyrrolidone, polyvinyl alcohol, and mixtures thereof,
wherein polyvinylpyrrolidone is especially preferred,
   in the composition (A) in a total amount of 0.5 to 14 wt %, preferably 1.0 to 11 wt %, especially preferably 2.0 to 10 wt %, with respect to the weight of the composition (A).

The present invention also relates to a method for cleaning and/or caring for keratin fibers, wherein the composition (A) for treating hair includes:
malic acid in an amount of 2 to 20 wt %, preferably 5 to 15 wt %, especially preferably 8 to 12 wt %, converted to the undissociated acid and with respect to the weight of the composition (A),
at least one of the amino acids arginine, histidine, or lysine and/or one salt thereof in a total amount of 0.4 to 7.0 wt %, preferably 0.8 to 5.0 wt %, especially preferably
1.5 to 4.0 wt %, converted to the undissociated acid and with respect to the weight of the composition (A),
50 to 92 wt %, especially preferably 60 to 87 wt %, and extraordinarily preferably 65 to 80 wt % of water, with respect to the weight of the composition (A), and
at least one polymer A, which has at least ten constitutive units of formula (I),

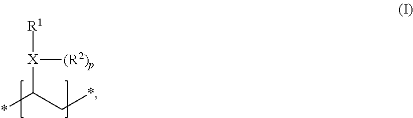

wherein
   X represents nitrogen or oxygen and
   R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
   p=0 if X represents oxygen and p=1 if X represents nitrogen,
wherein the polymer A does not include any permanently ionic constitutive units and is selected from polyvinylpyrrolidone, polyvinyl alcohol, and mixtures thereof, wherein polyvinylpyrrolidone is especially preferred,
   in the composition (A) in a total amount of 0.5 to 14 wt %, preferably 1.0 to 11 wt %, especially preferably 2.0 to 10 wt %, with respect to the weight of the composition (A).

The polymer (A) included in compositions (A) preferably used according to the invention has at least ten constitutive units of formula (I),

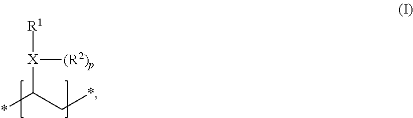

wherein
X represents nitrogen or oxygen and
R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
p=0 if X represents oxygen and p=1 if X represents nitrogen,
   wherein the polymer A does not include any permanently ionic constitutive units.

Surprisingly, it has been found that a polymer A, as described above and explained in more detail below, excellently supports the protective and reparative effect that the combination of at least one saturated dicarboxylic acid having 2 to 10 carbon atoms and/or at least one salt of said acid(s) and at least one amino acid of formula (VI), as explained above, has on damaged keratin fibers.

In the sense of the present invention, the term “polymer” is understood to mean polymers in the sense of the IUPAC definition that comprise at least 10 identical constitutive units.

According to the RÖMPP Chemie Lexikon, July 2009 edition, a substance composed of a collective of chemically uniformly constructed macromolecules (polymer molecules) is referred to as a polymer in accordance with a definition of the IUPAC, wherein said macromolecules or polymer molecules differ from each other in regard to the degree of polymerization, molar mass, and chain length. Thus, in the case of such polymerically uniform materials, all macromolecules are identically constructed and differ merely in the chain length thereof (degree of polymerization). According to this IUPAC definition, a polymer is furthermore a polyreaction product that is constructed of a plurality of molecules, in which one type or several types of atoms or atom groupings (constitutive units, basic building blocks, or repeat units) are strung together repetitively.

The number of constitutive units in a polymer is called the degree of polymerization. Polymers A and polymers B preferred according to the invention have a degree of polymerization in the range of 40 to 1000, preferably 100 to 800, especially preferably 350 to 650. Other polymers A preferred according to the invention having at least ten constitutive units of formula (I) include 40 to 1000, preferably 100 to 800, especially preferably 350 to 650 identical constitutive units of formula (I).

R¹ and R² preferably represent, independently of each other, hydrogen or a C₂-C₁₀ acyl group, which preferably selected from an acetyl, propanoyl, or n-butanoyl group, especially preferably from an acetyl group.

Polymers A preferred according to the invention have at least 10 constitutive units of formula (I), in which X represents nitrogen, wherein the polymer A does not include any permanently ionic constitutive units.

Other polymers A especially preferred according to the invention have at least 10 constitutive units of formula (I), in which X represents nitrogen and R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group.

If R¹ and R² form a five- or six-membered, saturated or unsaturated ring together with X, which ring optionally includes further heteroatoms, which are preferably selected from N and O, said ring is preferably substituted with at least one functional group, which is selected from ═O. An especially preferred substituent combination X, R² is a pyrrolidone group, a constitutive unit of formula (I) especially preferred according to the invention therefore being a unit of formula (Ia),

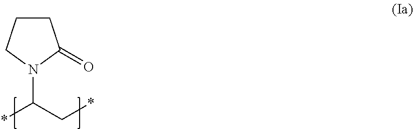

in which X represents nitrogen and R¹ and R² form a five-membered saturated ring together with this nitrogen atom, which ring does not include any further heteroatoms and is substituted with a functional group ═O in the 2 position.

Another especially preferred substituent combination X, R¹, R² is an ε-caprolactam group, a constitutive unit of formula (I) especially preferred according to the invention therefore being a unit of formula (I b),

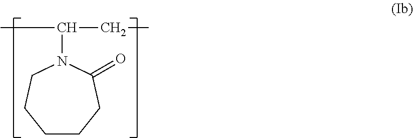

in which X represents nitrogen and R¹ and R² form a six-membered saturated ring together with this nitrogen atom, which ring does not include any further heteroatoms and is substituted with a functional group ═O.

Another especially preferred substituent combination X, R¹, R² is an imidazole group, another unit of formula (I) especially preferred according to the invention therefore being a unit in which X represents nitrogen and R¹ and R² form a five-membered unsaturated ring together with this nitrogen atom, which ring includes nitrogen as a further heteroatom.

Other polymers A preferred according to the invention have 25-100 mol %, preferably 55-100 mol %, especially preferably 85-100 mol % of constitutive units of formula (I), in which X represents nitrogen, wherein the polymer A does not include any permanently ionic constitutive units.

Other polymers A preferred according to the invention have 25-100 mol %, preferably 55-100 mol %, especially preferably 85-100 mol % of constitutive units of formula (I), in which X represents nitrogen and R¹ and R² form a five- or six-membered, saturated or unsaturated ring together with X, which ring optionally includes further heteroatoms, which are preferably selected from N and O, and is optionally substituted with at least one C1-C6 alkyl group and/or with at least one functional group, wherein the polymer A does not include any permanently ionic constitutive units.

Polymers A especially preferred according to the invention have 98-100 mol % of constitutive units of formula (Ia), wherein the polymer A does not include any permanently ionic constitutive units.

Polymers A extraordinarily preferred according to the invention have 98-100 mol % of constitutive units of formula (Ia) and have a degree of polymerization in the range of 40 to 1000, preferably 100 to 800, especially preferably 350 to 650, wherein the polymer A does not include any permanently ionic constitutive units. Especially preferred polymers A are polyvinylpyrrolidone homopolymers having a degree of polymerization in the range of 40 to 1000, preferably 100 to 800, especially preferably 350 to 650.

Another especially preferred substituent combination X, R¹, R² is a constitutive unit of formula (I), in which X represents oxygen, p is zero, and R¹ represents hydrogen.

Another especially preferred substituent combination X, R¹, R² is a constitutive unit of formula (I), in which X represents oxygen, p is zero, and R¹ represents an acetyl group.

Other polymers A preferred according to the invention include 75-92 mol % of constitutive units of formula (I), in which X represents oxygen, p is zero, and R¹ represents hydrogen, and 8-25 mol % of constitutive units of formula (I), in which X represents oxygen, p is zero, and R¹ represents an acetyl group, wherein the polymer A does not include any permanently ionic constitutive units.

Other polymers A preferred according to the invention include 40 to 1000, preferably 100 to 800, especially preferably 350 to 650 constitutive units of formula (I), thereof 75-92 mol % of constitutive units of formula (I), in which X represents oxygen, p is zero, and Rⁱ represents hydrogen, and 8-25 mol % of constitutive units of formula (I), in which X represents oxygen, p is zero, and R¹ represents an acetyl group, wherein the polymer A does not include any permanently ionic constitutive units.

Other polymers A preferred according to the invention include 65-25 mol % of constitutive units of formula (Ia) and 35-75 mol % of constitutive units of formula (I), in which X represents oxygen, p is zero, and R¹ represents an acetyl group, wherein the polymer A does not include any permanently ionic constitutive units.

Other polymers A preferred according to the invention include 40 to 1000, preferably 100 to 800, especially preferably 350 to 650 constitutive units of formula (I), thereof 65-25 mol % of constitutive units of formula (Ia) and 35-75 mol % of constitutive units of formula (I), in which X represents oxygen, p is zero, and R¹ represents an acetyl group, wherein the polymer A does not include any permanently ionic constitutive units.

The at least one polymer A having at least ten constitutive units of formula (I) does not have any permanent ionic charges. However, it is possible that the constitutive units of formula (I) exist in ionic form, particularly cationic form, for example as the result of the protonation of the nitrogen atom in an acidic carrier. But these charges are not permanent, but rather temporary, because these charges depend on the surrounding medium.

Preferred compositions (A) used according to the invention include at least one polymer A having at least ten constitutive units of formula (I).

Other preferred compositions (A) used according to the invention include the at least one polymer A having at least ten constitutive units of formula (I) in a total amount of 0.5 to 14 wt %, preferably 1.0 to 11 wt %, especially preferably 2.0 to 10 wt %, with respect to the weight of the composition (A), wherein the polymer A is selected from polyvinylpyrrolidone, polyvinyl alcohol, and mixtures thereof, wherein polyvinylpyrrolidone is especially preferred.

The carriers for the cleaning and/or care agents according to the invention can be solid, liquid, in the form of a gel, or pasty. Said carriers are preferably selected from aqueous systems, natural or synthetic oils, water-in-oil emulsions, or oil-in-water emulsions. Such systems and methods for the production thereof are known in the prior art, to which reference is hereby made. The preparations can be formulated as a cream, gel, or liquid. Furthermore, the agents can be formulated in the form of foam aerosols, which are introduced into aerosol containers having a foam valve together with a liquefied gas, such as propane-butane mixtures, nitrogen, CO₂, air, NO₂, dimethyl ether, hydrochlorofluorocarbon propellants, or mixtures thereof. The individual components of the method according to the invention are preferably used as a cream, gel, or liquid. Furthermore, the preparations used according to the invention can have two or more phases. Two-phase and multi-phase systems are systems comprising at least two separate, continuous phases. For example, an aqueous phase and one or more, e.g., two non-aqueous phases immiscible with each other can be present separate from each other in such systems. Also possible are, for example, a water-in-oil emulsion and an aqueous phase separated therefrom or a water-in-oil emulsion and an aqueous phase separated therefrom.

In another embodiment of the invention, the preparation can include a complexing agent, such as EDTA, NTA, β-alanine diacetic acid, a phosphonic acid, or mixtures of said substances.

Polyols, such as glycerol and partial glycerol ethers, particularly 3-[(2-ethylhexyl)oxy]-1,2-propanediol, 2-ethyl-1,3-hexanediol, 1,3-butanediol, 1,4-butanediol, 1,2-propanediol, 1,3-propanediol, pentanediols, such as 1,2-pentanediol, hexanediols, such as 1,2-hexanediol or 1, 6-hexanediol, dodecanediol, particularly 1,2-dodecanediol, neopentylglycol, and ethylene glycol, are suitable as further active substances. In particular, 2-ethyl-1,3-hexanediol, 1,2-propanediol, 1,3-propanediol, and 1,3-butanediol have proven especially well suited.

These polyols are included in the preparations according to the invention and the preparations used according to the invention preferably in a total amount of 0.1-10, particularly 0.5-5 wt %, with respect to the entire preparation.

Of course, alcohols miscible with water only to a limited extent can also be used according to the invention, particularly if multi-phase systems should be obtained.

“Miscible with water to a limited extent” is understood to mean alcohols that have a solubility in water at 20° C. of no more than 10 wt %, with respect to the water mass.

Linear and/or branched, saturated and/or unsaturated fatty acids having 6-30 carbon atoms can be used as fatty acids in amounts of 0.1-15 wt %, with respect to the entire agent. The fatty acids are monocarboxylic acids.

Furthermore, it has been found that polymers are advantageously used in the context of the method according to the invention. Therefore, in a preferred embodiment, polymers are added to the preparations used according to the invention, wherein cationic, anionic, amphoteric, and non-ionic polymers have proven effective.

The term “cationic polymers” is understood to mean polymers that have a “temporarily” or “permanently” cationic group in the main chain and/or side chain. According to the invention, polymers that have a cationic group regardless of the pH value of the preparation are called “permanently cationic”. They are generally polymers that include a quaternary nitrogen atom, for example in the form of an ammonium group. Preferred cationic groups are quaternary ammonium groups. In particular, polymers in which the quaternary ammonium groups are bonded to a polymer main chain constructed of acrylic acid, methacrylic acid, or derivatives thereof by means of a C1-4 hydrocarbon group have proven especially suitable.

Homopolymers of general formula (IX),

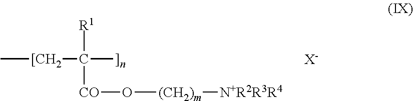

in which R¹=—H or —CH₃, R², R³, and R⁴ are selected independently of each other from C1-4 alkyl, alkenyl, or hydroxyalkyl groups, m=1, 2, 3, or 4, n is a natural number, and X⁻ is a physiologically acceptable organic or inorganic anion, and copolymers consisting largely of the monomer units indicated in formula (II) and non-ionogenic monomer units are especially preferred cationic polymers. In the context of these polymers, those for which at least one of the following conditions applies are preferred according to the invention:
R¹ represents a methyl group.
R², R³, and R⁴ represent methyl groups.
m has a value of 2.

Halide ions, sulfate ions, phosphate ions, methosulfate ions, and organic ions such as lactate, citrate, tartrate, and acetate ions, for example, are considered as physiologically acceptable counterions X⁻. Halide ions, particularly chloride, are preferred.

An especially suitable homopolymer is poly(methacryloyloxyethyl trimethylammonium chloride), which is cross-linked if desired and has the INCI name Polyquaternium-37. If desired, the cross-linking can be accomplished by means of olefinically polyunsaturated compounds, such as divinylbenzene, tetraallyloxyethane, methylenebisacrylamide, diallyl ether, polyallyl polyglyceryl ether, or allyl ethers of sugars or sugar derivatives such as erythritol, pentaerythritol, arabitol, mannitol, sorbitol, sucrose, or glucose. Methylenebisacrylamide is a preferred cross-linking agent.

The homopolymer is preferably used in the form of a non-aqueous polymer dispersion, which should have a polymer fraction not below 30 wt %. Such polymer dispersions are commercially available under the names Salcare® SC 95 (approximately 50% polymer fraction, additional components: mineral oil (INCI name: Mineral Oil) and tridecyl polyoxypropylene polyoxyethylene ether (INCI name: PPG-1-Trideceth-6)) and Salcare® SC 96 (approximately 50% polymer fraction, additional components: mixture of diesters of propylene glycol with a mixture of caprylic acid and capric acid (INCI name: Propylene Glycol Dicaprylate/Dicaprate) and tridecyl polyoxypropylene polyoxyethylene ether (INCI name: PPG-1-Trideceth-6)).

Copolymers having monomer units according to formula (II) preferably include acrylamide, methacrylamide, acrylic acid C₁₋₄ alkyl esters, and methacrylic acid C₁₋₄ alkyl esters as non-ionogenic monomer units. Among these non-ionogenic monomers, acrylamide is especially preferred. These copolymers, as in the case of the homopolymers described above, can be cross-linked. A copolymer preferred according to the invention is the cross-linked acrylamide/methacryloyloxyethyl trimethylammonium chloride copolymer. Such copolymers in which the monomers are present in a weight ratio of approximately 20:80 are commercially available as an approximately 50% non-aqueous polymer dispersion under the name Salcare® SC 92.

Additionally preferred cationic polymers are, for example:
quaternized cellulose derivatives, which are commercially available under the names Celquat® and Polymer JR®. The compounds Celquat® H 100, Celquat® L 200, and Polymer JR® 400 are preferred quaternized cellulose derivatives.
cationic alkyl polyglycosides according to patent document DE 4413686,
cationized honey, such as the commercial product Honeyquat® 50,
cationic guar derivatives, such as the products sold under the trade names Cosmedia® Guar and Jaguar® in particular,
polysiloxanes having quaternary groups, such as the commercially available products Q2-7224 (manufacturer: Dow Corning; a stabilized trimethylsilylamodimethicone), Dow Corning® 929 Emulsion (including a hydroxylamine-modified silicone, which is also referred to as amodimethicone), SM-2059 (manufacturer: General Electric), SLM-55067 (manufacturer: Wacker), and Abil®-Quat 3270 and 3272 (manufacturer: Evonik; diquaternary polydimethylsiloxanes, Quaternium-80),
polymeric dimethyldiallylammonium salts and copolymers thereof with esters and amides of acrylic acid and methacrylic acid. The products commercially available under the names Merquat® 100 (poly(dimethyldiallylammonium chloride)) and Merquat® 550 (dimethyldiallylammoniumchloride/acrylamide copolymer) are examples of such cationic polymers.
copolymers of vinylpyrrolidone with quaternized derivatives of dialkylaminoalkyl acrylate and methacrylate, such as vinylpyrrolidone/dimethylaminoethyl methacrylate copolymers quaternized with diethyl sulfate. Such compounds are commercially available under the names Gafquat® 734 and Gafquat® 755.
vinylpyrrolidone/vinylimidazolium methochloride copolymers, which are offered under the names Luviquat® FC 370, FC 550, FC 905, and HM 552,
quaternized polyvinyl alcohol,
and the polymers having quaternary nitrogen atoms in the polymer main chain known under the names Polyquaternium 2, Polyquaternium 17, Polyquaternium 18, and Polyquaternium 27.

The polymers known under the names Polyquaternium-24 (commercial product, e.g., Quatrisoft® LM 200) likewise can be used as cationic polymers. The copolymers of vinylpyrrolidone, which are available as commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370, likewise can be used according to the invention.

Other cationic polymers that can be used according to the invention are the “temporarily cationic” polymers. Said polymers typically include an amino group which, at certain pH values, exists as a quaternary ammonium group and thus is cationic. Preferred are chitosan and salts thereof, which are freely available commercially, for example under the trade names Hydagen® CMF, Hydagen® HCMF, Kytamer® PC, and Chitolam® NB/101.

Cationic polymers preferred according to the invention are cationic cellulose derivatives and chitosan and derivatives thereof, particularly the commercial products Polymer® JR 400, Hydagen® HCMF, and Kytamer® PC, cationic guar derivatives, cationic honey derivatives, particularly the commercial product Honeyquat 50, cationic alkyl polyglycosides according to patent document DE 4413686, and polymers of the type Polyquaternium-37.

The use of vitamins, provitamins, and vitamin precursors and derivatives thereof has likewise proven advantageous.

According to the invention, vitamins, provitamins, and vitamin precursors that are typically assigned to the groups A, B, C, E, F, and H are preferred.

Retinol (vitamin A₁) and 3,4-didehydroretinol (vitamin A₂) belong to the group of substances referred to as vitamin A. β-carotene is the provitamin of retinol. For example, vitamin A acid and esters thereof, vitamin A aldehyde, and vitamin A alcohol and esters thereof such as the palmitate and the acetate are considered as a vitamin A component according to the invention. The preparations used according to the invention include the vitamin A component preferably in amounts of 0.05-1 wt %, with respect to the entire preparation.

The vitamin B group or the vitamin B complex includes, among other things:
Vitamin B₁ (thiamine)
Vitamin B₂ (riboflavin)
Vitamin B₃. This designation is often used to refer to the compounds nicotinic acid and nicotinic acid amide (niacinamide). Preferred according to the invention is nicotinic acid amide, which is included in the preparations used according to the invention preferably in amounts of 0.05 to 1 wt %, with respect to the entire preparation.
Vitamin B₅ (pantothenic acid, panthenol, and pantolactone). In the context of this group, panthenol and/or pantolactone is preferably used. Derivatives of panthenol that can be used according to the invention are, in particular, the esters and ethers of panthenol and cationically derivatized panthenols. Individual representatives are, for example, panthenol triacetate, panthenol monoethyl ether and the monoacetate thereof, and the cationic panthenol derivatives disclosed in WO 92/13829. The mentioned compounds of the vitamin B₅ type are included in the agents according to the invention preferably in amounts of 0.05-10 wt %, with respect to the entire agent. Amounts of 0.1-5 wt % are especially preferred.
Vitamin B₆ (pyridoxine, pyridoxamine, and pyridoxal).
Vitamin C (ascorbic acid). Vitamin C is included in the agents according to the invention preferably in an amount of 0.1 to 3 wt %, with respect to the entire agent. Use in the form of palmitic acid ester, glucosides, or phosphates can be preferred. Use in combination with tocopherols can likewise be preferred.
Vitamin E (tocopherols, particularly α-tocopherol). Tocopherol and derivatives thereof, under which, in particular, the esters and the acetate, the nicotinate, the phosphate, and the succinate fall, are included in the agents according to the invention preferably in amounts of 0.05-1 wt %, with respect to the entire agent.
Vitamin F. The term “vitamin F” is typically understood to mean essential fatty acids, particularly linoleic acid, linolenic acid, and arachidonic acid.
Vitamin H. The compound (3aS,4S,6aR)-2-oxohexahydrothienol[3,4-d]imidazole-4-valeric acid is referred to as vitamin H, but in the meantime the trivial name biotin has become established for said compound. Biotin is included in the agents according to the invention preferably in an amount of 0.0001 to 1.0 wt %, particularly in an amount of 0.001 to 0.01 wt %, with respect to the weight of the entire agent.

The agents according to the invention preferably include vitamins, provitamins, and vitamin precursors from the groups A, B, E, and H. Panthenol, pantolactone, pyridoxine and derivatives thereof, nicotinic acid amide, and biotin are especially preferred.

Finally, plant extracts can be included in the agents according to the invention.

Furthermore, silicone gums and cationic silicones, such as the commercially available products Q2-7224 (manufacturer: Dow Corning; a stabilized trimethylsilylamodimethicone), Dow Corning® 939 Emulsion (including a hydroxylamine-modified silicone, which is also referred to as amodimethicone), SM-2059 (manufacturer: General Electric), SLM-55067 (manufacturer: Wacker), and Abil®-Quat 3270 and 3272 (manufacturer: Evonik; diquaternary polydimethylsiloxanes, Quaternium-80), are suitable as conditioning active substances. A suitable anionic silicone is the product Dow Corning® 1784.

Further auxiliary substances and additives are, for example:
thickeners such as agar-agar, guar gum, alginates, xanthan gum, gum arabic, gum karaya, locust bean gum, linseed gums, dextranes, cellulose derivatives, e.g., methyl cellulose, hydroxyalkyl cellulose, and carboxymethyl cellulose, starch fractions and derivatives such as amylose, amylopectin, and dextrins, or clays such as bentonite,
perfume oils,
solvents and solubilizers such as ethanol, isopropanol, ethylene glycol, propylene glycol, glycerol, and diethylene glycol,
pearlizing agents such as ethylene glycol mono- and distearate and PEG-3 distearate,
pigments,
propellants such as propane-butane mixtures, N₂O, dimethyl ether, CO₂, and air,
antioxidants.

### EXAMPLES

The following agents according to the invention were produced (amount specifications in wt %):

a) Color-protection shampoos that repair the hair structure

| | Shampoo | Shampoo | Shampoo |
|---|---|---|---|
| | 1 | 2 | 3 |
| Sodium Laureth Sulfate | 11.00 | 11.00 | 10.00 |
| Cocamidopropyl Betaine | 1.00 | 1.50 | 3.00 |
| Disodium Cocoamphodiacetate | 0.50 | — | — |
| Cocamide MEA | 0.50 | 0.50 | — |
| PEG-12 Dimethicone | 0.50 | 0.30 | — |
| Glycol Distearate | 1.20 | — | — |
| PEG-7 Glyceryl Cocoate | 0.40 | 0.60 | 0.80 |
| Polyquaternium-10 | 0.90 | — | 0.60 |
| Guar Hydroxypropyltrimonium | — | 0.60 | — |
| Chloride | | | |
| Panthenol | 0.30 | 0.20 | 0.20 |
| Polyvinylpyrrolidone | — | 1.0 | 2.0 |
| Dimethicone (50 cSt/25° C.) | 0.10 | — | 0.50 |
| Hydrogenated Castor Oil | 0.20 | 0.10 | 0.30 |
| Succinic Acid | 0.3 | 0.5 | 0.1 |
| Disodium succinate | 0.9 | 0.7 | 0.4 |
| Arginine | 0.2 | 0.1 | 0.4 |
| Lysine hydrochloride | 0.2 | 0.3 | 0.1 |
| 3-[(2-ethylhexyl)oxy]-1,2- | 0.9 | 0.9 | 0.9 |
| propanediol | | | |
| Phenoxyethanol | 0.9 | 0.9 | 0.9 |
| Perfume | 0.8 | 0.8 | 0.8 |
| Water | Ad 100 | Ad 100 | Ad 100 |

After repeated cleaning (10-20 cleaning processes) with the aforementioned shampoos, oxidatively dyed hair has a brilliant hair color, the fastness properties of which do not substantially change. In addition, the hair washed with the aforementioned shampoos has a visually appealing shine and an especially soft texture.

b) Rinse-off hair-conditioning composition (conditioner; amounts in wt %)

| | 1 | 2 | 3 | Conditioner 4 |
|---|---|---|---|---|
| Cetearyl Alcohol | 2.50 | 6.00 | 9.00 | 9.00 |
| Quaternium-87 | 2.00 | — | — | — |
| Quaternium-91 | — | 0.4 | — | — |
| Propylene Glycol | 0.50 | 0.50 | 1.00 | 1.00 |
| Isopropyl Myristate | 0.30 | 0.30 | 0.50 | 0.50 |
| Distearoylethyl | 1.50 | 0.50 | 1.0 | 1.0 |
| Hydroxyethylmonium | | | | |
| Methosulfate | | | | |
| 3-[(2-ethylhexyl)oxy]-1,2- | 0.40 | 0.40 | 0.60 | 0.60 |
| propanediol | | | | |
| Phenoxyethanol | 0.40 | 0.40 | 0.60 | 0.60 |
| Stearamidopropyl | — | — | 1.20 | 1.20 |
| Dimethylamine | | | | |
| Polyvinylpyrrolidone | 1.0 | 0.1 | 1.5 | 1.5 |
| Silicone Quaternium-1 | — | 0.05 | — | — |
| Silicone Quaternium-6 | — | — | 0.05 | 0.05 |
| Polyquaternium-37 | 0.20 | — | — | — |
| Dicaprylyl Carbonate | 0.20 | — | — | — |
| Panthenol | 0.10 | 0.10 | 0.10 | 0.10 |
| Benzophenone-4 | 0.05 | 0.05 | 0.05 | 0.05 |
| Amodimethicone/Morpho- | 0.02 | — | — | — |
| linomethyl Silsesquioxane | | | | |
| Copolymer | | | | |
| Hydrolyzed Keratin | 0.01 | 0.01 | 0.01 | 0.01 |
| Succinic Acid | 0.5 | 0.5 | 0.3 | — |
| Disodium succinate | 0.8 | 0.7 | 0.5 | — |
| DL-malic acid | — | — | — | 0.5 |
| Arginine | 0.2 | 0.1 | 0.05 | 0.05 |
| Lysine hydrochloride | 0.2 | 0.1 | 0.05 | 0.05 |
| Perfume | 0.25 | 0.25 | 0.25 | 0.25 |
| Water | Ad 100 | Ad 100 | Ad 100 | Ad 100 |

c) Method according to the invention for restructuring hair cleaning with a shampoo according to the invention

The following compositions (A) were provided (amount specifications in wt %):

| | Composition (A-1) | Composition (A-2) |
|---|---|---|
| 3-[(2-ethylhexyl)oxy]-1,2- | 0.40 | 0.40 |
| propanediol | | |
| Phenoxyethanol | 0.40 | 0.40 |
| Polyvinylpyrrolidone | 10.0 | 10.0 |
| Hydrolyzed Keratin | 0.01 | 0.01 |
| Succinic Acid | 4.50 | — |
| Disodium succinate | 9.50 | — |
| DL-malic acid | — | 4.5 |
| Arginine | 3.0 | 3.0 |
| Lysine hydrochloride | 3.0 | 3.0 |
| Potassium sorbate | 0.001 | 0.001 |
| Water | Ad 100 | Ad 100 |

The following shampoo composition (B-1) was provided (amount specifications in wt %):

| | Shampoo (B- |
|---|---|
| | 1) |
| Sodium Laureth Sulfate | 11.00 |
| Cocamidopropyl Betaine | 1.00 |
| Disodium Cocoamphodiacetate | 0.50 |
| Cocamide MEA | 0.50 |
| PEG-12 Dimethicone | 0.50 |
| Glycol Distearate | 1.20 |
| PEG-7 Glyceryl Cocoate | 0.40 |
| Polyquaternium-10 | 0.90 |
| Panthenol | 0.30 |
| Dimethicone (50 cSt/25° C.) | 0.10 |
| Hydrogenated Castor Oil | 0.20 |
| 3-[(2-ethylhexyl)oxy]-1,2- | 0.9 |
| propanediol | |
| Phenoxyethanol | 0.9 |
| Perfume | 0.8 |
| Water | Ad 100 |

5 grams of the composition (A-1) were mixed with 50 grams of the shampoo (B-1); the mixture was then applied to wetted hair of the head, massaged in for 0.5 minutes, and thereafter rinsed out with water.

5 grams of the composition (A-2) were mixed with 50 grams of the shampoo (B-1); the mixture was then applied to wetted hair of the head, massaged in for 0.5 minutes, and thereafter rinsed out with water.

d) Method according to the invention for restructuring hair care with a rinse-off hair-conditioning composition according to the invention

The following rinse-off hair-conditioning composition (B-2) was provided (amounts in wt %):

| | |
|---|---|
| Cetearyl Alcohol | 6.00 |
| Quaternium-91 | 0.20 |
| Propylene Glycol | 0.50 |
| Isopropyl Myristate | 0.30 |
| Distearoylethyl | 1.50 |
| Hydroxyethylmonium Methosulfate | |
| 3-[(2-ethylhexyl)oxy]-1,2- | 0.40 |
| propanediol | |
| Phenoxyethanol | 0.40 |
| Panthenol | 0.10 |
| Benzophenone-4 | 0.05 |
| Perfume | 0.25 |
| Water | Ad 100 |

5 grams of the composition (A-1) were mixed with 50 grams of the hair-conditioning composition (B-2); the mixture was then applied to wetted hair of the head, massaged in for 0.5 minutes, allowed to act for 1 minute, and thereafter rinsed out with water.

5 grams of the composition (A-2) were mixed with 50 grams of the hair-conditioning composition (B-2); the mixture was then applied to wetted hair of the head, massaged in for 0.5 minutes, allowed to act for 1 minute, and thereafter rinsed out with water.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent for cleaning and/or caring for keratin fibers, which is free of peroxide compounds, comprising:
a) at least one saturated dicarboxylic acid having 2 to 10 carbon atoms and/or at least one salt of said acid(s),
b) at least one amino acid of formula (VI) 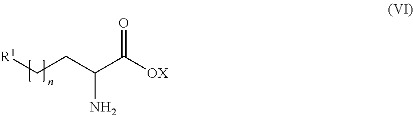
wherein
X represents a hydrogen atom or a mono- or divalent cation;
n represents 0, 1, 2, or 3;
R¹ represents a residue that is selected from an amino group, a guanidine group, a (1H-imidazol-4-yl) group, a carboxylic acid amide group —CONH₂, a 1H-indol-3-yl group, a thiol group —SH, and a methylsulfanyl group —SCH₃, or at least one salt of said amino acid, and
c) at least one cleaning and/or care active substance, selected from surfactants, fatty alcohols, and oils.

2. The cleaning or care agent according to claim 1, wherein the at least one saturated dicarboxylic acid having 2 to 10 carbon atoms is selected from the group consisting of succinic acid, malic acid, oxalic acid, malonic acid, adipic acid, pimelic acid, suberic acid, azelaic acid, sebacic acid, and mixtures of these acids.

3. The cleaning or care agent according to claim 1, wherein the at least one saturated dicarboxylic acid having 2 to 10 carbon atoms is selected from the group consisting succinic acid, malic acid, and mixtures thereof.

4. The cleaning or care agent according to claim 1, wherein the at least one dicarboxylic acid having 2 to 10 carbon atoms is succinic acid.

5. The cleaning or care agent according to claim 1, wherein the least one saturated dicarboxylic acid having 2 to 10 carbon atoms is included in a total amount of 0.05 to 6 wt % converted to the undissociated acid and with respect to the weight of the cleaning and/or care agent.

6. The cleaning or care agent according to claim 1, wherein the least one saturated dicarboxylic acid having 2 to 10 carbon atoms is included in a total amount of 0.1 to 4 wt % converted to the undissociated acid and with respect to the weight of the cleaning and/or care agent.

7. The cleaning or care agent according to claim 1, wherein the least one saturated dicarboxylic acid having 2 to 10 carbon atoms is included in a total amount of 0.5 to 2.5 wt % converted to the undissociated acid and with respect to the weight of the cleaning and/or care agent.

8. The cleaning or care agent according to claim 1, wherein the at least one amino acid of formula (VI) is selected from arginine, lysine, histidine, asparagine, glutamine, cysteine, methionine, tryptophan, and mixtures thereof.

9. The cleaning or care agent according to claim 1, wherein the at least one amino acid of formula (VI) includes arginine and lysine.

10. The cleaning or care agent according to claim 1, wherein the at least one amino acid of formula (VI) is included in a total amount of 0.05 to 3 wt % converted to the undissociated acid and with respect to the weight of the cleaning or care agent.

11. The cleaning or care agent according to claim 1, wherein the at least one amino acid of formula (VI) is included in a total amount of 0.3 to 1.2 wt % converted to the undissociated acid and with respect to the weight of the cleaning or care agent.

12. The cleaning or care agent according to one of claim 1, further comprising at least one compound of general formula (III) is included, 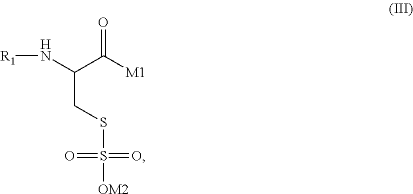
wherein
R1 represents a hydrogen atom or a structural element of formula (IV), 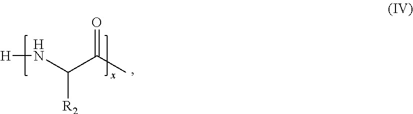
wherein
x represents an integer from 1 to 100,
the residue R2 in each of the structural elements of formula (IV) can be selected independently of the preceding structural element of formula (IV),
R2 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group,
M1 represents the group —OM2 or a structural element of formula (V), 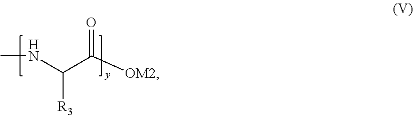
wherein
y represents an integer from 1 to 100,
the residue R3 in each of the structural elements of formula (V) can be selected independently of the preceding structural element of formula (V),
R3 represents a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group, or a (sulfosulfanyl)methyl group,
M2 represents a hydrogen atom, an equivalent of a mono- or polyvalent cation, or an ammonium ion (NH₄)⁻,
wherein one or more compounds of formula (III) stated above are preferably included in a total amount of 0.001 to 2.5 wt %, more preferably 0.005 to 0.2 wt %, and especially preferably 0.01 to 0.07 wt %, with respect to the weight of the cleaning and/or care agent according to the invention.

13. The cleaning or care agent according to claim 1, further comprising at least one polymer A is included, which has at least ten constitutive units of formula (I), 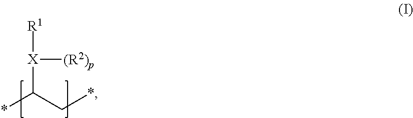
wherein
X represents nitrogen or oxygen and
R¹ and R² represent, independently of each other, hydrogen or a C2-C10 acyl group or R¹ and R² form a five- or six-membered, saturated or unsaturated ring together with X, which ring optionally includes further heteroatoms, which are preferably selected from N and O, and/or is optionally substituted with at least one C1-C6 alkyl group and/or with at least one functional group, and
p=0 if X represents oxygen and p=1 if X represents nitrogen,
wherein the polymer A does not include any permanently ionic constitutive units, wherein the at least one polymer A having at least ten constitutive units of formula (I) is included preferably in a total amount of 0.2 to 5 wt %, especially preferably 0.5 to 3 wt %, extraordinarily preferably 1.0 to 2.3 wt %, with respect to the weight of the cleaning or care agent.

14. The cleaning or care agent according to claim 13, wherein the at least one polymer A having at least ten constitutive units of formula (I) is selected from polymers that have 98-100 mol % of constitutive units of formula (Ia), 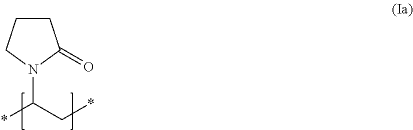
and have a degree of polymerization in the range of 40 to 1000, and
wherein the polymer A does not include any permanently ionic constitutive units.

15. A method for cleaning and/or caring for keratin fibers, particularly human hair, wherein a cleaning or care agent according to claim 1 is applied to the keratin fibers, particularly to the human hair, and optionally rinsed out after an exposure time of 0.1 to 60 minutes.
